(19) United States
(12) Patent Application Publication
(10) Pub. No.: US 2025/0345005 A1
(43) Pub. Date: Nov. 13, 2025

(54) MACHINE LEARNING FOR AGGREGATING AND EVALUATING DATA FROM A SENSOR ENABLED ENVIRONMENT

(71) Applicant: LogicMark, Inc., Louisville, KY (US)

(72) Inventors: Chia-Lin Simmons, Moraga, CA (US); Rafael Saavedra, Cupertino, CA (US); Peter Williams, Belmont, CA (US)

(21) Appl. No.: 19/272,488

(22) Filed: Jul. 17, 2025

Related U.S. Application Data
(63) Continuation-in-part of application No. 19/089,555, filed on Mar. 25, 2025.
(60) Provisional application No. 63/569,575, filed on Mar. 25, 2024.

(51) Int. Cl. A61B 5/00

(52) U.S. Cl. CPC A61B 5/7264; A61B 5/7275

(57) ABSTRACT

Machine learning for aggregating and evaluating data from a sensor enabled environment (SEE) may be provided by receiving first sensor data from a (SEE in which a person under monitoring (PUM) is monitored, wherein the first sensor data are received from a first configuration of the SEE, wherein the SEE comprises environmental sensors and an artificial intelligence or machine learning (AI/ML) model; identifying, via the AI/ML model analyzing the first sensor data, a first behavioral, health, wellness, or safety (BHWS) event occurring in the SEE affecting the PUM; in response to identifying the first BHWS event, reconfiguring how the SEE monitors the PUM from the first configuration to a second configuration based on the first BHWS event; receiving second sensor data from the SEE according to the second configuration; and identifying, via analysis of the second sensor data, a second BHWS event occurring in the SEE affecting the PUM.

### CROSS-REFERENCES TO RELATED APPLICATIONS

The present disclosure is continuation in part of U.S. patent application Ser. No. 19/089,555, titled “MACHINE LEARNING FOR AGGREGATING AND EVALUATING DATA FROM A SENSOR ENABLED ENVIRONMENT”, which was filed on 2025 Mar. 25, which claims benefit of U.S. Provisional Patent Application No. 63/569,575, titled “MACHINE LEARNING FOR AGGREGATING AND EVALUATING DATA FROM A SENSOR ENABLED ENVIRONMENT”, which was filed on 2024 Mar. 25, and each of which is incorporated herein in its entirety.

### BACKGROUND

Sensor-enabled environments may include one or more fixed location sensors, devices or systems installed in an environment or one or more mobile sensors, devices or systems that are present in the environment, all of which can be initialized, calibrated or configured for monitoring the health and wellness of one or more person under monitoring (PUM) in that environment. Data from a sensor-enabled environment may be processed to determine whether one or more health events has occurred or is likely to occur.

### SUMMARY

Systems, methods, and apparatuses are provided for training and implementation of a machine learning model for aggregating and evaluating data from a sensor enabled environment (SEE) for health and wellness care management for one or more persons under monitoring (PUM). In an example, a system comprises a sensor-enabled environment, a memory, and a processing device configured to receive data from the sensor enabled environment, align the data with at least one pattern framework indicative of a behavior of a person under monitoring, evaluate the at least one pattern framework to detect or predict a wellness event, and send an alert indicative of the detected wellness event.

In another example, a method comprises receiving data from the sensor enabled environment, aligning the data with at least one pattern framework indicative of a behavior of a person under monitoring, evaluating the at least one pattern framework to detect or predict a wellness event, and sending an alert indicative of the detected wellness event.

In another example, systems, methods, and apparatuses are provided for: receiving first sensor data from a sensor enabled environment (SEE) in which a person under monitoring (PUM) is monitored, wherein the first sensor data are received from a first configuration of the SEE, wherein the SEE comprises a plurality of environmental sensors and an artificial intelligence or machine learning (AI/ML) model; identifying, via the AI/ML model analyzing the first sensor data, a first behavioral, health, wellness, or safety (BHWS) event occurring in the SEE affecting the PUM; in response to identifying the first BHWS event, reconfiguring how the SEE monitors the PUM from the first configuration to a second configuration based on the first BHWS event; receiving second sensor data from the SEE according to the second configuration; and identifying, via analysis of the second sensor data, a second BHWS event occurring in the SEE affecting the PUM.

In some such examples, reconfiguring the SEE from the first configuration to the second configuration includes performing a reconfiguration selected from the group consisting of: (A) switching the AI/ML model to a second AI/ML model; and analyzing the second sensor data with the second AI/ML model as part of identifying the second BHWS event, (B) reconfiguring at least one of the plurality of environmental sensors, wherein the second sensor data are received, from the SEE, at least in part, using the at least one of environmental sensors that has been reconfigured; and (C) changing how data received from individual environmental sensors in the plurality of environmental sensors are prepared for analysis by the AI/ML model.

In some such examples, switching the AI/ML model used to analyze the first sensor data to the second AI/ML model to analyze the second sensor data comprises: using a first model from a group consisting of an Environment Awareness Model, a Pattern Model, and a meta-context model as the AI/ML model to process the first sensor data; and using a second model, different from the first model from the group consisting of the Environment Awareness Model, the Pattern Model, and the meta-context model to process the second sensor data; wherein the second one of the Environment Awareness Model, the Pattern Model, and the meta-context model is selected based on: a type of the BHWS event detected.

In some such examples, reconfiguring the at least one of the plurality of environmental sensors includes sending a configuration command for the at least one of the plurality of environmental sensors from the group consisting of: activating the at least one of the plurality of environmental sensors; deactivating the at least one of the plurality of environmental sensors; and increasing a granularity of data collected by the at least one of the plurality of environmental sensors; decreasing the granularity of data collected by the at least one of the plurality of environmental sensors; increasing a reporting rate of the at least one of the plurality of environmental sensors; decreasing the reporting rate of the at least one of the plurality of environmental sensors; and changing an optical focus of the at least one of the plurality of environmental sensors.

In some such examples, reconfiguring how data received from individual environmental sensors of the plurality of environmental sensors are amalgamated for analysis by the AI/ML model in the second sensor data relative to the first sensor data is selected according to a segmentation scheme selected from the group consisting of: identifying second features from the second sensor data that are not identified from the first sensor data, wherein the second features are present in the first sensor data; analyzing longer segments of the second sensor data compared to the first sensor data; analyzing shorter segments of the second sensor data compared to the first sensor data; and incorporating additional data from a second environmental sensor of the plurality of environmental sensors with the second sensor data that was not incorporated with the first sensor data.

In some such examples, reconfiguring the SEE from the first configuration to the second configuration based on the first BHWS event includes: wherein different data sharing policies are associated with different types of BHWS event, the method further comprising: identifying a type of the BHWS event detected; and selecting the second configuration according to a data sharing policy associated with the type of the BHWS event.

In some such examples, identifying the first BHWS event further comprises: detecting a state of the PUM or the SEE; and analyzing the state using an Environment Awareness Model.

In some such examples, identifying the first BHWS event further comprises: detecting a series of states of the PUM or the SEE via the Environment Awareness Model; and analyzing the series of states using a Pattern Model.

In some such examples, identifying the first BHWS event further comprises: detecting a behavioral pattern via the analyzed series of states; and analyzing the behavior using a meta-context model in comparison to at least one of a health care profile (HCP), model in a personalized physics engine (PPE), or a learned habitual behavior of the PUM.

In some such examples, the systems, methods, and apparatuses use a Large Language Model or Large Context Model artificial intelligence or machine learning (LLM/LCM AI/ML) system to predict a future BHWS event via tokens or concepts of previous BHWS events included in the first sensor data and the second sensor data.

In some such examples, the systems, methods, and apparatuses use a Large Language Model or Large Context Model artificial intelligence or machine learning (LLM/LCM AI/ML) system to identify that the first BHWS event is incongruous to the second BHWS event according to tokens or concepts represented by the first BHWS event and the second BHWS event with respect to a quiescent state of the PUM or SEE.

In some such examples, the systems, methods, and apparatuses also compare the first BHWS event against the second BHWS event to confirm whether the first BHWS occurred or is a hallucination; and in response to confirming via identification of the second BHWS event that the first BHWS actually occurred, transmit a notification to a stakeholder for care of the PUM that identifies occurrence of the actual event.

In some such examples, the first BHWS event is a predicted event generated by the AI/ML model, and the systems, methods, and apparatuses also: compare the predicted event against a model of physical capabilities of the PUM in a personalized physics engine (PPE); in response to determining that at least one behavior included in the predicted event is within the physical capabilities of the PUM according to the model in the PPE: transmit a notification to a stakeholder for care of the PUM that identifies the predicted event; and select the second configuration of the SEE to capture data in a format for recording an actual occurrence of the predicted event.

In some such examples, the first BHWS event is a predicted event generated by the AI/ML model, and the systems, methods, and apparatuses also: compare the predicted event against a model of physical capabilities of the PUM in a personalized physics engine (PPE); and in response to determining that at least one behavior included in the predicted event is outside of the physical capabilities of the PUM according to the model in the PPE, classify the predicted event as a hallucination of the AI/ML model.

In some such examples, the systems, methods, and apparatuses also: generate a first token that includes a type of the first BHWS event in an unencrypted format and a segment of the first sensor data used by the AI/ML model to identify the first BHWS event in an encrypted format; and transmit the first token to a first external system.

In some such examples, the external system is selected from the group consisting of: a distributed or blockchain ledger; and a stakeholder device or system.

In some such examples, the stakeholder device or system is associated with a stakeholder for care of the PUM selected from the group consisting of: the PUM; a caregiver of the PUM; a friend of the PUM; a neighbor of the PUM; a family member of the PUM; an insurance provider for the PUM; a medical professional; and an emergency responder.

In some such examples, the systems, methods, and apparatuses also: transmit the first token to a second external system, wherein the second external system is provided a decryption schema for the encrypted format that is not provided to the first external system.

In some such examples, the systems, methods, and apparatuses also: identify a second external system associated with a second decryption schema based on the type of the first BHWS event and a type of the second BHWS event; generate a second token that includes the first type of the first BHWS event and a second type of the second BHWS event in the unencrypted format and a second segment of the second sensor data used by the AI/ML model to identify the second BHWS event in a second encrypted format decryptable according to the second decryption schema; and transmit the second token to a second external system.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the Figures and the Detailed Description. Moreover, it should be noted that the language used in this specification has been principally selected for readability and instructional purposes, and not to limit the scope of the inventive subject matter.

### BRIEF DESCRIPTION OF THE DRAWINGS

The description will be more fully understood with reference to the following figures, which are presented as exemplary aspects of the disclosure and should not be construed as a complete recitation of the scope of the disclosure, wherein:

FIG. 1 illustrates a block diagram of an example contextual categorization and classification data flow, according to example embodiments of the present disclosure.

FIG. 2 illustrates a block diagram of an example system performing categorization and classification, according to example embodiments of the present disclosure.

FIG. 3 illustrates a block diagram of an example machine learning system, according to example embodiments of the present disclosure.

FIG. 4 illustrates a block diagram of a token management dataflow, according to example embodiments of the present disclosure.

FIG. 5 illustrates a block diagram of a state management dataflow, according to example embodiments of the present disclosure.

FIG. 6 illustrates a block diagram of an attention and focus dataflow, according to example embodiments of the present disclosure.

FIG. 7 illustrates a block diagram of a token management dataflow for responses, according to example embodiments of the present disclosure.

FIG. 8 illustrates a flowchart for an example method for monitoring a SEE, according to example embodiments of the present disclosure.

FIG. 9 is a flowchart of an example method for generating and maintaining a linguistic AI/ML model for machine learning for aggregating and evaluating data from a sensor enabled environment, according to embodiments of the present disclosure.

FIG. 10 is a flowchart of an example method for deploying and using a linguistic AI/ML model for machine learning for aggregating and evaluating data from a sensor enabled environment.

FIG. 11 illustrates a block diagram of an example segmentation flow, according to example embodiments of the present disclosure.

FIG. 12 is a flowchart of an example method for processing data from a SEE, according to example embodiments of the present disclosure.

FIG. 13 illustrates an example computing device, as may be used as a controller in a SEE to monitor a PUM, as part of a sensor monitoring a PUM, as part of a central or distributed service providing calibration systems for generating and curating AI/ML models for distribution to the SEEs, and the like, according to embodiments of the present disclosure.

### DETAILED DESCRIPTION

Techniques are disclosed herein for training and implementation of a machine learning model for aggregating and evaluating data from a sensor enabled environment (SEE) for health and wellness care management for one or more persons under monitoring (PUM). Monitoring of some individuals may be desirable for the health, wellbeing, and personal safety of those individuals. This is particularly the case for elderly individuals who may have limited memory, for example. Such monitoring, however, introduces significant challenges regarding scalability and quality of life.

Existing techniques for monitoring individuals for health reasons typically require than an individual relocate to a facility equipped with personnel and equipment specialized to perform such monitoring. Besides the obvious loss of freedom that this entails, monitoring for certain conditions can be quite invasive, and patient maltreatment can be a chronic problem for such facilities. One potential solution to this problem is placement of sensors around an individual's living spaces to remotely monitor that individual. This strategy poses additional problems, however. Namely, a single monitored individual may generate exceptionally large quantities of data which can be difficult to monitor to a satisfactory level. Employing large numbers of people to monitor this data would be impractical, especially since much of the monitoring time is uneventful to such a degree that proper attention may be difficult to maintain. It is therefore desirable to implement a system which can automatically aggregate and evaluate data from one or more SEE at scale.

Systems and methods of the present disclosure achieve this aim by training a machine learning model to recognize a variety of patterns in data that may be produced by a sensor-enabled environment. This machine learning model may align this data with one or more corresponding pattern frameworks, which may then be used to determine one or more corresponding behaviors of a person under monitoring (PUM). Once the one or more behaviors have been determined, the machine learning model may correlate the one or more behaviors with likely health or wellness events which are occurring or which may soon occur to the PUM. The machine learning model may send an alert indicative of the health or wellness event to monitoring personnel, thereby potentially limiting human involvement to one or more PUM who may be in need of additional attention or intervention.

A SEE may generate one or more data sets from one or more sensors, devices, or systems deployed or present within the SEE. These data sets may be arranged into one or more patterns. These pattern arrangements may, in whole or in part, be undertaken at an “edge”. For example, these pattern arrangements may be determined by the one or more sensors, devices, or systems or by a receiving system, for example a hub, in any arrangement. In some example embodiments, these patterns may be formulated in pattern frameworks based at least in part on behaviors of one or more PUM as they undertake their daily activities or routines. These patterns may in turn form contextual behaviors that can, at least in part, represent a PUM that is domiciled in such a SEE as they engage in their daily activities and life.

Each of these patterns may be specified in differing arrangements. For example, they may be arranged according to one or more categorizations including but not limited to an ontology, taxonomy, or other organization. These categorizations may be based on a number of factors, and in some embodiments may be generated by one or more artificial intelligence or machine learning (AI/ML) modules. In some embodiments, these categorizations may represent the behaviors of a PUM in a SEE and may be considered or evaluated in and across multiple categorizations including, for example, spatial, temporal, contextual, health, or wellness.

In some embodiments, a pattern may span more than one of these categorizations or may contribute to or comprise another one or more categories or categorizations, for example those generated through the use of one or more AI/ML modules using one or more training data sets including, for example, the data sets or patterns generated by one or more SEE.

Using these example categorizations, a PUM's behaviors may be represented in the form of spatial (i.e. locational), temporal (i.e. in or over a time period), contextual behavior (i.e. the current activity of the PUM), or health and wellness (i.e. including health and wellness monitoring). This example categorization comprising patterns may include the data sets generated by, at least in part, the one or more sensors, devices, or systems deployed or present in a SEE where the PUM or other stakeholders are under monitoring.

The relationships between the categories, the dimensions, or feature sets thereof may be deterministic. An example of this may be sensors that are located in specific areas may form part of a spatial group or category, such as a bedroom or similar. The relationships between the categories, the dimensions, or feature sets thereof may also be non-deterministic. An example of this may be a set of sensors that generate data based on haptics, such as foot fall and the like.

Each of these categories may have one or more dimensions or one or more feature sets which may comprise such data sets or the patterns in any arrangement. In some embodiments, one or more topological representations may be used to manage such dimensions or feature sets including, for example, in one or more repositories of patterns, dimensions, or features.

This holistic representation of a SEE and the one or more PUM domiciled therein enables a comprehensive monitoring approach that, based on the various data sets or patterns, may be calibrated and optimized to identify variations in the context and behaviors of a PUM that may have a health and wellness impact across various time periods, in various locations, or in the context of their overall wellness and health.

One of the aspects of this approach may be the use of AI/ML in combination with digital twins and including in some embodiments one or more physics engines such that variations in the overall PUM wellness and health may be identified in a detailed responsive ongoing or timely manner. This approach enables and supports a more proactive approach to the identification, detection, mitigation, or alleviation of the health and wellness issues that challenge us all as we age.

The use of this combination of SEE, AI/ML, digital twins or physics engines in combination may provide significant benefits for the PUM, whilst respecting their privacy, quality of life and their inherent life choices.

The categories described herein may be evaluated in isolation or in any combination to create differing perspectives on the PUM overall health conditions. For example, if a PUM has limited movement, such as issues with their mobility, the care hub systems may be calibrated to account for an uneven footfall of that PUM and in consequence the dimensions of the spatial categorizations, for example, may be adjusted accordingly.

An aspect of this approach may be identification of structures comprising patterns, categorizations, dimensions, or features represented, for example, as multi-dimensional manifolds in an efficient manner, where an outline of the structure is evident with minimal possible data sets. For example, the use of convolutional neural networks in combination with recurrent neural networks may create a minimal structure based on these data sets. This may include the use, for example, of large language models (LLM).

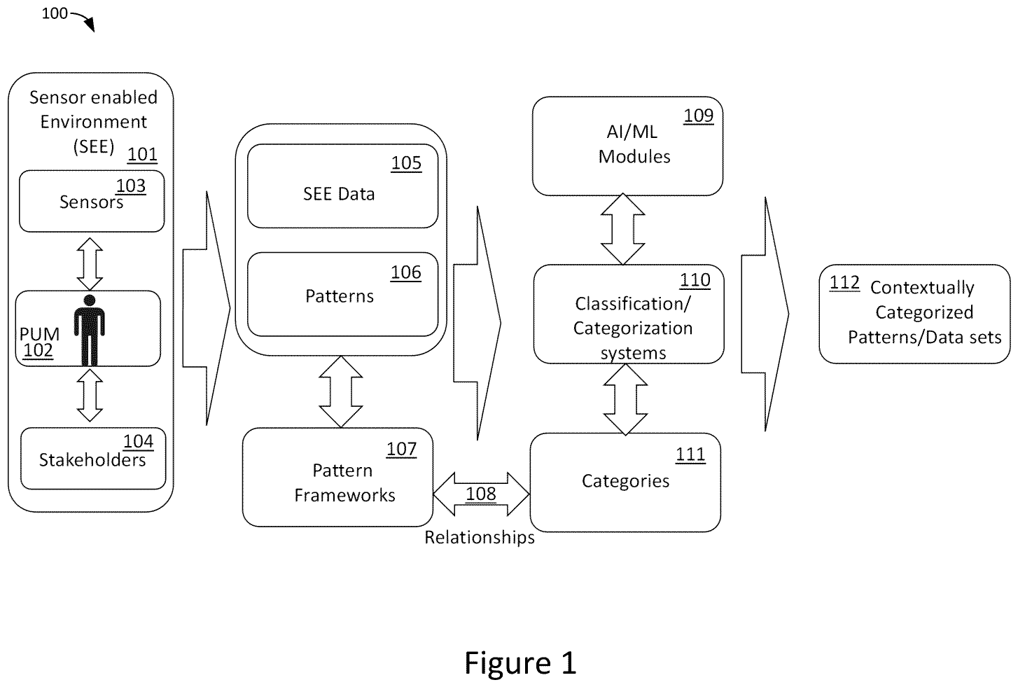

FIG. 1 illustrates a block diagram of an example contextual categorization and classification data flow 100, according to example embodiments of the present disclosure. In this example, a SEE 101 includes sensors, devices, or systems (generally referred to as sensors 103) for monitoring a PUM 102 or stakeholders 104. These sensors 103 generate data sets 105 representing SEE 101, PUM 102, and Stakeholder 104 activities. These data sets 105 may be aligned to pattern frameworks 107 to generate patterns 106 comprising, at least in part, SEE data sets 105 and Pattern Frameworks 107. The SEE data sets 105 or patterns 106 may be communicated to one or more classification/categorization systems 110. These systems 110 may use one or more categories 111 or one or more AI/ML modules 109 to align SEE data sets 105 and patterns 106 to generate contextually categorized patterns or data sets (generally referred to as classifications 112). In some embodiments, relationships 108 between, for example, pattern frameworks 107 and categories 111 may be persisted in one or more repositories such as in a graph database.

In some embodiments, a set of categorizations may be used to configure the patterns of data generated by the SEE into actionable arrangements that may be employed for managing the health and wellbeing of the PUM. This categorization may provide the basis for one or more techniques for algorithmic processing of these categories and the patterns or data sets they include so as to create one or more responses, including but not limited to response candidates, that may be deployed.

The patterns or data sets and their formulation into categories may involve the use of one or more AI/ML systems where, for example, these patterns or data sets may form one or more training sets for such AI/ML modules.

Several of these initial example categorizations are outlined herein, however there may be additional categorizations that are generated by the one or more AI/ML modules or created by initialization, calibration, or configuration of the one or more sensors, devices, or systems of the SEE or the one or more hub systems in any arrangement.

A spatial category may be location centric, for example a room in an environment, a portion of a room, a space around a room, or encompassing a feature of a room such as a sofa, or a function, such as a food preparation area. The spatial category may include one or more volumetric metrics based on a location, where boundary of the spatial domain may be determined, at least in part, by the boundaries of the space, such as the dimensions of a room.

In some embodiments, there may be a spatial category that is centered on the PUM such as, for example, a spatial domain with a diameter minimum of “an arms length”, representing a reach of the PUM or a “legs length” representing their stride, both of which may be related to PUM specifications such as a height, weight, gait, leg/arm dimensions, or other metrics. For example, this may include a typical 1 to 1.5 Meter circular diameter based on a body center line and having a height based upon the PUM's height plus, for example, 1 meter.

In this manner the spatial categorization of the PUM may be evaluated in relation to other spatial aspects of the environment, for example to ascertain when the PUM may interact with another spatial entity. This may be particularly useful in determining potential impacts for a PUM when navigating an environment.

Temporal categorizations may include 24 hour clock time or time periods related to the one or more behaviors of the PUM, including those of the one or more contextual behaviors of the PUM. The 24 hour clock time may be used to segment the activities of a PUM into, for example, sleeping, exercising, eating, and other activities.

Contextual behaviors may include one or more pattern frameworks that have, for example, been deployed as part of a calibration of a SEE. These contextual behaviors may include one or more data sets generated by the one or more sensors, devices, or systems which are aligned with one or more pattern frameworks to create a contextual behavior that is specific to a PUM. This approach may also be applied to one or more other stakeholders with whom the PUM interacts, for example a carer who may undertake food preparation for the PUM.

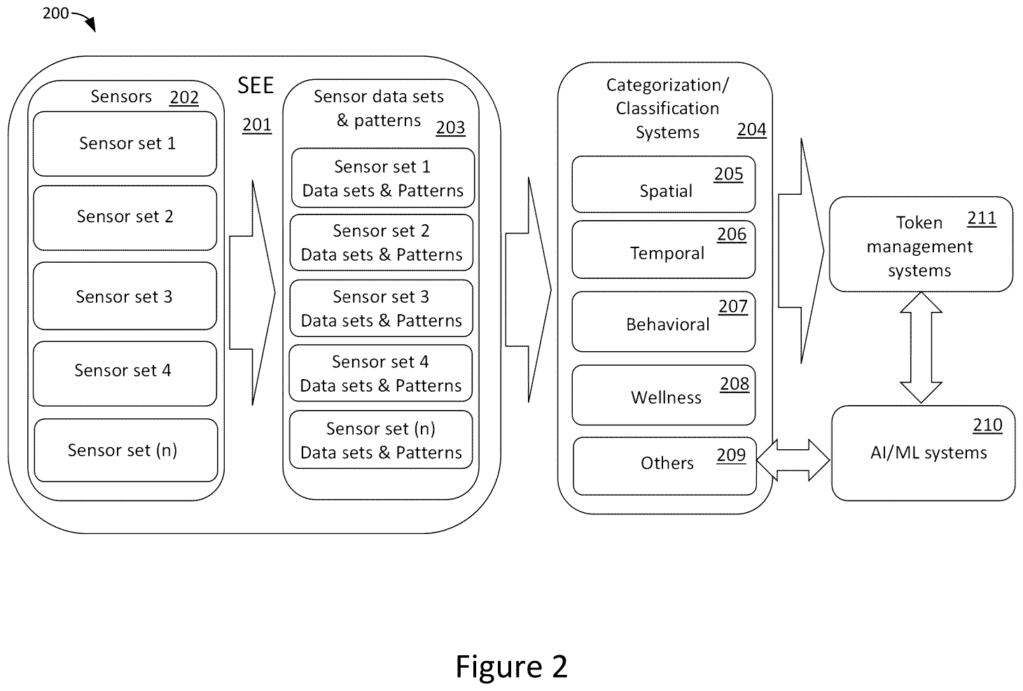

FIG. 2 illustrates a block diagram of an example system 200 performing categorization and classification, according to example embodiments of the present disclosure. A SEE 201 may comprise a set of sensors 202 in any arrangement, which may generate sensor data sets or patterns 203. These sensors 202, data, sets or patterns 203 may be communicated to one or more categorization/classification systems 204 where, for example, one or more categorization/classification may be undertaken. Each of these may include data about the one or more sets of sensors 202, the data sets, or patterns they generate 203 and their relationships to the SEE 201 in any arrangement. For example, categorization/classification systems may include spatial categories 205, temporal categories 206, behavioral categories 207, wellness categories 208, or other categories 209, which for example may be identified by one or more AI/ML systems 210. These categorizations or classifications may be communicated to one or more token management systems 211, where they may be formed into one or more tokenized representations.

The health and wellness categorizations may comprise, at least in part, a healthcare profile (HCP) of the PUM insofar as the HCP specifies health and wellness reasons for monitoring. The health and wellness categorizations may include, for example one or more health and wellness events, such as those detrimental to a PUM (e.g. a fall) where the data sets or patterns generated by the one or more sensors, devices, or systems of a SEE are arranged in one or more pattern frameworks representing such an event. These event pattern frameworks may include, for example, falls, breathing difficulties, mobility difficulties, heart or other organ difficulties, injuries (such as cutting with a knife when preparing food), or other events. In some embodiments these event pattern frameworks may be prioritized based, at least in part, on the specific of the reasons for the monitoring, represented by the HCP. In this manner, such operations as attention or focus processing as described herein may be prioritized to those most likely event frameworks.

These categorizations may comprise data sets, patterns, features, or dimensions that are specific to that categorization. For example, a temporal category may include a timeline dimension representing previous, current, and future time periods.

In some embodiments, there may be dimensions or feature sets that span one or more categorizations, such as those that involve spatial, temporal, or behavioral elements. For example, this may include quality of life dimensions such as those associated with diet, exercise, or hobbies.

In some embodiments there may be further categorizations that are formulated, at least in part, by the one or more AI/ML modules. This may include a determination by such modules of feature sets, dimensions, or other characteristics of spatial, temporal, behavioral, or wellness categories as well as further categories that may be generated by the AI/ML modules.

In some embodiments, these further categories may be evaluated, for example, in one or more digital twins in collaboration in some circumstances with one or more physics engines or one or more sets of specifications, possibly including the HCP, to determine, at least in part, an accuracy, reliability, or utility of the specifications. This can in some embodiments include evaluation by one or more human actors, including the PUM or other stakeholders.

In some embodiments a framework, such as those that form, at least in part, one or more categorizations, may involve one or more digital twins which may be initialized with data sets or patterns derived from one or more calibrated sensors or repository-based data sets from similar situations. These digital twins may have an associated set of strategies that represent behaviors of PUM from this initial state. These may be aligned with game theory, vectors, topologies, or other representations. These strategies may represent the most likely behavior variations, based at least in part on the SEE data sets, AI/ML modules, game theory strategies, or physics engines.

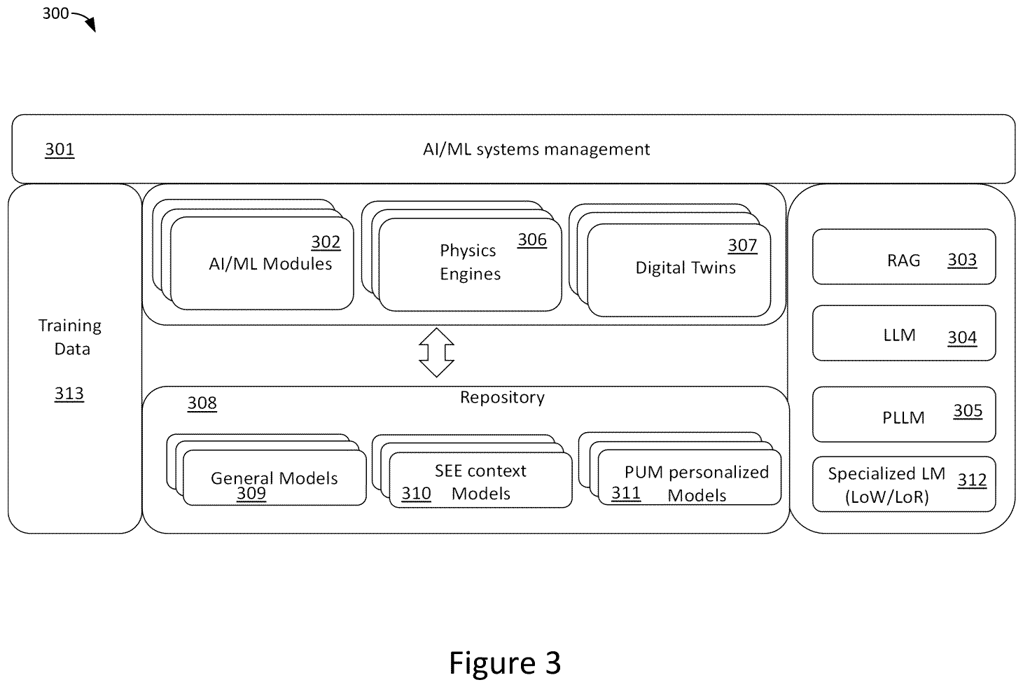

FIG. 3 illustrates a block diagram of an example machine learning system 300, according to example embodiments of the present disclosure. In this example an AI/ML management system 301 comprises or manages one or more AI/ML modules 302, one or more physics engines 306, or one or more digital twins 307 in any arrangement. These AI/ML modules 302, physics engines 306 or digital twins 307 may generate, based on one or more sets of training data 313 including, for example, SEE data sets 105, patterns 106, or categorizations/classifications 112, some of which may be represented by one or more tokens, to create one or models held in one or more repositories 308. For example, such models may include general models 309, SEE context models 310, or PUM personalized models 311. In some embodiments one or more AI/ML systems may be employed, such as retrieval augmented generation (RAG) AI 303, large language model (LLM) AI 304, private large language model (PLLM) AI 305, or one or more specialized language model (LM), such as one or more care language (Language of wellness/language of care response) 312.

In some embodiments, there may be one or more languages representing care of one or more PUM in one or more SEE. These languages may be used for communication between one or more sensors, devices, or systems, including but not limited to care hubs or care processing systems, and may, in whole or in part, support communications between machines or humans in any arrangement.

Such languages may form specialized representations of context, events, actions, situations, or other characteristics of one or more PUM domiciled or present in one or more SEE. In some embodiments these languages may comprise sets of tokens representing various care conditions and states thereof, and may include spatial, temporal, behavioral, or wellness categorizations of the one or more data sets or patterns generated by the one or more sensors, devices, or systems of one or more SEE. For example, such categorizations may include relationships between and amongst the one or more sensors, devices, or systems, including care hubs and care processing systems, and the data sets or patterns they generate.

In some embodiments one or more token management systems may be employed for the generation of one or more tokens representing, for example, sensors, devices, or systems or the data sets or patterns they generate. In the same manner, pattern frameworks, categorizations, classifications, or relationships between these entities may also be formed into one or more tokens by one or more token management systems. The token management systems may employ one or more cryptographic techniques that may be applied to one or more tokens in support of one or more security, privacy, or distribution schemas which can, for example, form part of one or more care hub or care processing systems.

These tokenized representations may, for example, in whole or in part, be aligned with one or more large language models, including private or specialized language models. For example, a carer may be able to, using one or more care languages, query a wellness state of a PUM.

In some embodiments, for example, a care language may be instantiated as a language of wellness (LoW), and may be employed by a sensor, device, or system communication means for one or more machines, including but not limited to other sensors, devices, or systems, including, for example, care hubs or care processing systems with, at least in part, a purpose of monitoring a wellness and health of a person under care in a sensor enabled environment.

The one or more data sets or patterns that may be generated by the one or more sensors, devices, or systems embedded or present in a SEE may, in some embodiments, be represented in the form of a language of wellness (LoW). This language, in common with other languages, may have a set of expressions, a syntax, and a set of semantics. For example, the expressions may comprise representations of one or more data sets or patterns that are tokenized versions of those expressions. In some embodiments, tokens representing data sets or patterns from one or more sensors, devices, or systems may be used in combination to form one or more further tokens representing one or more patterns.

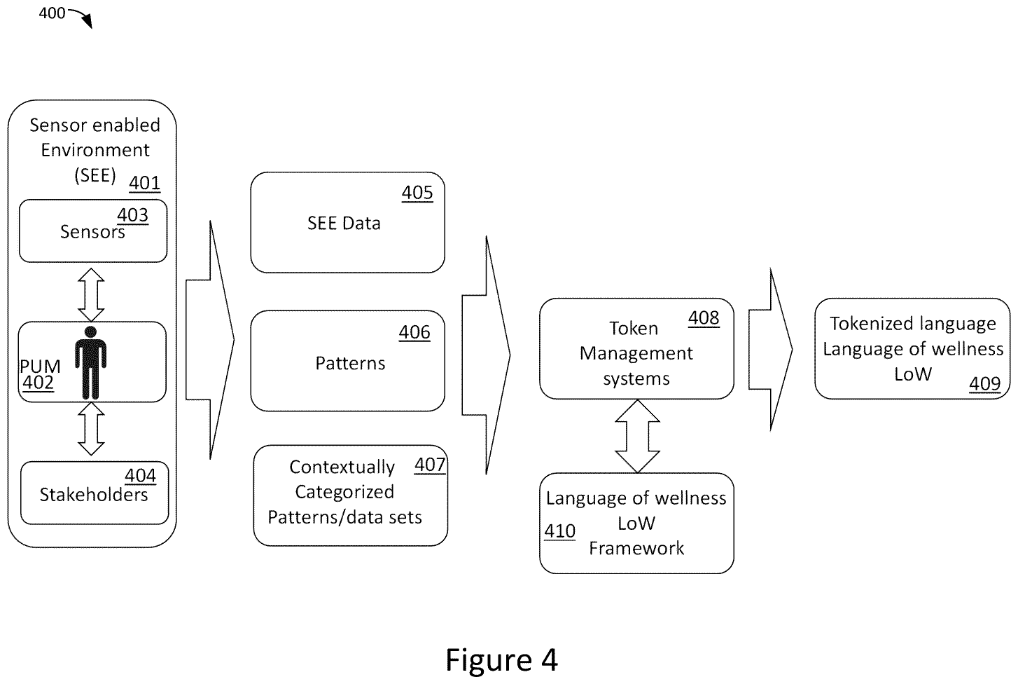

FIG. 4 illustrates a block diagram of a token management dataflow 400, according to example embodiments of the present disclosure. For example a SEE 401 may include one or more PUM 402 or one or more stakeholders 404 which are monitored by one or more sensors, devices, or systems 403. The data sets 405, patterns 406, or contextually categorized patterns or data sets 407 generated by such SEE 401 may be communicated to one or more token management systems 408 to generate, for example using a language framework 410, one or more tokenized care languages, for example a language of wellness (LoW) 409.

For example, a pattern framework may comprise a set of tokens which may be open or closed. In some embodiments, a pattern may comprise, for example, token N out of a set of Y tokens, where N is a subset of Y. In this example, a pattern token (N) may be specified as an algorithm where certain tokens representing data sets from the one or more sensors, devices, or systems in a SEE are arranged in a specific combination.

The tokenized patterns or data sets may form the language of wellness (LoW), whereby the language may include tokens that are created, at least in part, by one or more ML/AI systems and may form part of a model developed by such an AI/ML module that is, in part or in whole, trained on the data sets or patterns of the one or more sensors, devices, or systems of one or more SEE.

Tokenization enables processing of data sets into arrangements representing combinations. Some examples of this may include but are not limited to motion detection, haptic footfall detection, audio detection, or other detections which may be combined to represent, for example, a PUM moving from one location in an environment to another. In this example the token or set thereof may be part of a spatial categorization (e.g. moving from spatial location A to spatial location B), a temporal categorization (e.g. at time T for duration D), a contextual behavior (e.g. moving from a couch corresponding to spatial location A to a kitchen area corresponding to spatial location B) for morning coffee, or a wellness and health categorization (e.g. footfall and audio sensor data sets outside parameter thresholds, potentially indicating the PUM having mobility difficulty).

Tokenization may be employed where, for example, a token may include a set of segments comprising one or more data sets from one or more sensors, devices, or systems or other sources including other tokens in any arrangement. Tokens may also include one or more thresholds or one or more relationships with one or more patterns. These token segments may be available to one or more sensors, devices, systems, care hub services, or care processing services, for example using one or more cryptographic key regimes or other access control paradigms.

In some embodiments a monitoring language may include one or more known health and wellness events, including those that are detrimental or beneficial to a PUM, which may be represented by patterns, data sets, or tokens.

A language comprising a set of tokens may in some embodiments be processed by a Large Language Model where the tokens, forming such language, are processed by the LLM such that a context of any one token is constrained by the model or capabilities of the LLM, such that this context is, at least in part, determined by such processing.

In some embodiments, the language of wellness (LoW) may include one or more categorizations which may represent a syntax of the language. These categorizations may include, for example, spatial, temporal, behavioral, or health and wellness. This syntax may be extensible where, for example, one or more AI/ML systems may generate, based on one or models created from one or more training data sets comprising the data sets or patterns of the one or more sensors, devices, or systems of one or more SEE, further categorizations or classifications that are represented as tokens and form part of the syntax of the language.

In some embodiments a relationship between categories may be determined by one or more care hubs or care processing systems such that a set of categories (e.g. spatial, temporal, behavioral, or health and wellness) are given priority through, for example, specification, declaration, weighting, or similar over further categories that are generated by the one or more AI/ML modules. In some embodiments one or more physics engines may be employed to evaluate the one or more categories generated by the one or more AI/ML modules to ensure, at least in part, that the categories comply with applicable physics of the configured physics engine.

In some embodiments, there may be ontologies or taxonomies of these categories, as well as other organizational arrangements.

In some embodiments certain sensors, devices, or systems may represent data sets or patterns thereof in specific tokens. For example, a time measuring sensor may generate temporal tokens or a haptic sensor may generate haptic tokens. However, these tokens may be combined and synchronized to reflect sensor capabilities that may have common characteristics. In some embodiments, this may include but is not limited to identity, time, or location.

In some embodiments a care hub or care processing system may be configured such that specific sensors, devices, or systems have specified relationships with one or more categories. This may include specifying one or more sensors, devices, or systems as providers of tokens representing the data sets or patterns generated by such one or more sensors, devices, or systems as forming part of a particular spatial, temporal, behavioral, wellness, or other categorization or classification that can, at least in part, form part of a language of wellness (LoW).

In some embodiments, the raw data sets generated by the one or more sensors, devices, or systems may form training data sets for one or more AI/ML modules. This may be the case when an AI/ML module is tasked with generating models that are independent of any foreknowledge, such as pre-existing, specified, or deterministic categorizations or classifications. In this example, unsupervised learning techniques may be applied.

In some embodiments, state, including context represented by one or more data sets, patterns, categorizations, or classifications, may be tokenized and form part of the LoW. For example, in some embodiments, there may be certain elements of LoW that represent state or context such as, for example, temporal elements including past, current (including time periods), future, spatial elements, such as locational, one or more contextual behaviors derived, at least in part, form one or more pattern frameworks, wellness and health elements, for example those based, at least in part, on event frameworks.

In some embodiments, a LoW may enable differing perspectives using AI/ML models that represent these differing perspectives. An example of these differing perspectives may be those of differing stakeholders who interact with a PUM.

In some embodiments a private large language model (PLLM) may be employed to generate one or more models representing behaviors or interactions of a PUM in a SEE. For example, the PLLM may use the data sets or patterns generated by the one or more sensors, devices, or systems of a SEE to generate, at least in part, one or more models representing the PUM in a SEE, for example using a language of wellness (LoW).

In some embodiments the relationship between PUM behaviors represented, for example, by one or more contextual behavior and state may, at least in part, be determined by a language of wellness (LoW).

Such a language, comprising a tokenized representation of the state of the PUM in a SEE, may be aligned with one or more of these states. In some embodiments such sates may be quantized such that a specific set of data generated by the one or more sensors, devices or systems employed or present in the SEE, represented as patterns, for example, may form one or more quantized states.

In some embodiments, this quantization of state may be based, at least in part, on one or more categorizations comprising a categorization and one or more variables expressed in some embodiments as metrics, which may be employed as weightings representing these variables and where state may be a specific balance between each of the variables in the form of a ratio forming a topological representation of one or more dimensions such that state may be represented in some embodiments as an enclosed space within a topology. Each of these spaces may have a shape and may vary depending on the data sets, patterns, or weight variables of dimensions that form boundaries of the shape.

State shapes may be formalized as a set of classified entities including, for example, quiescent states where a state shape may change based, at least in part, on the data sets or patterns of the sensors, devices, or systems forming the SEE. In some embodiments, these changes may be expressed as vectors, which may include a rate of change (velocity), and a rate of rate of change (acceleration).

Each of these vectors may be used, at least in part, by one or more AI/ML systems to predict likely state shapes which represent the data sets or patterns that in turn represent PUM behaviors. For example, each state shape may have a finite set of potential shape shifts where the vectors represent relationships between these shapes.

In some embodiments one or more AI/ML modules may be employed in combination with one or more Digital Twins to generate one or more models representing potential shape shifts, one or more state, state changes, state transitions, or other state characteristics of the PUM domiciled in a SEE.

In some embodiments these shapes may be compared and evaluated from multiple PUM, where the identity of the PUM is not discernable from the state shape while the state shape remains effective to, at least in part, calculate likely vectors based on aggregated set of shapes representing a diaspora of PUM with similar HCP.

For example, a dimension may be expressed as a vector from a point which may be an origination or an intersection of one or more other dimensions. In this manner a vector of each dimension may have a differing trajectory such that an angle between the vectors may represent a degree of alignment of the dimensions. For example, if the angle between the dimension and the vector is acute, then the alignment of the dimensions may be evaluated to determine, at least in part, one or more metrics that may represent a quality of life (QoL) of the PUM in their SEE domicile as these dimensions form part of the representation of the state of that PUM in their SEE.

A set of metrics may be employed by one or more AI/ML models as representations of QoL of a PUM, and may be used, at least in part, as an element of an improvement strategy. This may be expressed, for example, in the form of game theory strategies where payoffs include metrics representing the quality of life of the PUM.

These strategies may include predictive and deterministic elements where, for example, the deterministic elements are based, at least in part, on the data set representations from the SEE of the domiciled PUM. The predictive elements may be based, at least in part, on game strategies that include state shapes where the associated vectors represent potential changes to those states quantized in the form of game theory strategies. In some embodiments this may include the deployment of AI/ML modules and one or more physics engines to, at least in part, ensure that the predicted vectors are within characteristics and capabilities of the PUM in their SEE enabled domicile.

In some embodiments, temporal dimensions may have vectors that may be forward (predictive) looking from a current time or rear looking (historical). In either case, a vector perpendicular to a temporal dimension may be indicative of multiple parallel dimensions within the state space.

In some embodiments, the state of one or more categorizations including but not limited to contextual behaviors, spatial, temporal, or wellness and health may be evaluated on a continuous or sampled basis to determine, at least in part, an earliest indication of a health or wellness event affecting the PUM. This evaluation can, in some embodiments, include one or more verifications that the early indications, represented by a change in state, are confirmed by multiple sensors, devices, or systems in a manner configured to reduce any false positives.

These combinations of patterns, data sets, or tokens may be evaluated to ascertain that the change in state conforms to one or more known patterns, data sets, or token relationships which, in whole or in part, may represent a health and wellness event, for example a fall.

In some embodiments, one or more topologies may be employed, in combination with one or more digital twins, to, at least in part, generate one or more predictive representations of the one or more states of the SEE, including the PUM therein.

For example, state shapes comprising patterns arranged into spatial, temporal, behavioral, or wellness based categorized data sets may be used in one or more digital twins where one or more characteristics of these patterns may be varied to, at least in part, determine thresholds for each dimension (spatial/temporal/behavioral/wellness), including the relationships between them.

In some embodiments, one or more AI/ML modules may be used to modulate the digital twin data sets that are derived, at least in part, from the SEE data sets. These modulations may be within boundaries that are representations of the possible data sets that one or more sensors, devices, or systems may generate in a SEE. The modulations may be in multiple dimensions and may involve correlated feature sets, that is to say if one feature is prominent in a particular modulation, then another may degenerated in response. The modulations may be temporal or may be based, at least in part, on contextual behaviors of a PUM, including health and wellness events. For example, a modulation may be based on a recuring PUM wellness behavior, for example a cough, which may be aligned to a seasonal occurrence (e.g. an allergy).

For example, if pattern A data set A1 becomes data set A2, which for example may be determined, at least in part by such data exceeding one or more thresholds, this may indicate a change in state. Such state change can, for example, be communicated to one or more AI/ML modules for evaluation including but not limited to extrapolation, for example using one or more digital twins to generate one or more sets of possible new states. These new states may be validated by one or more physics engines or one or more sets of specifications (e.g. HCP). These potential states can, for example, be compared or validated against other known patterns, such as those stored in one or more repository, or are identified as new patterns or states. These new patterns or states may include health and wellness events.

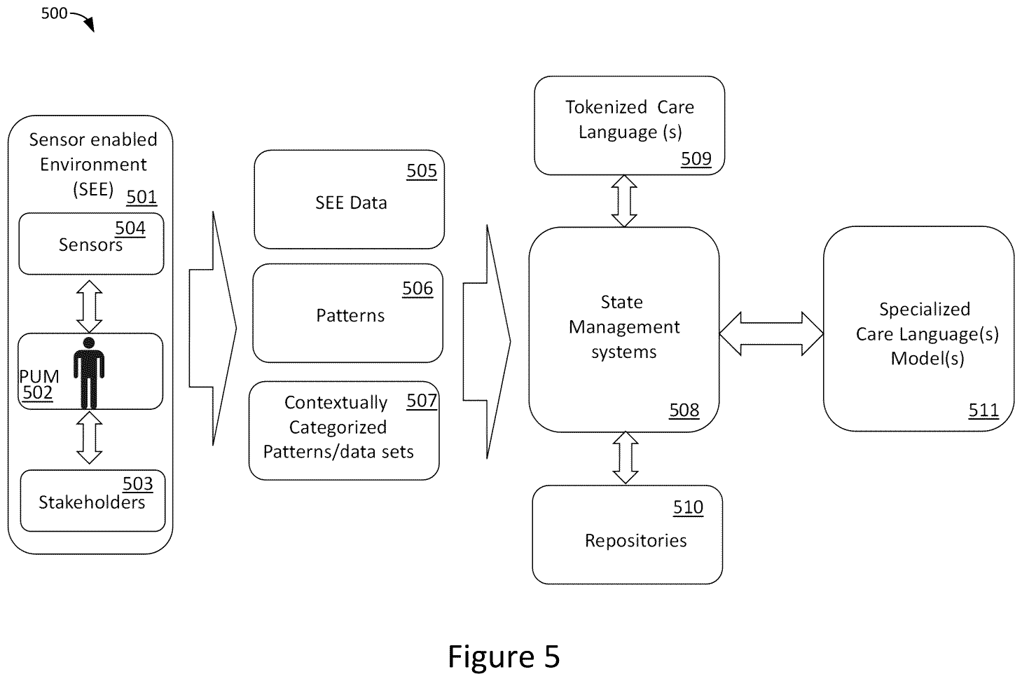

FIG. 5 illustrates a block diagram of a state management dataflow 500, according to example embodiments of the present disclosure. A SEE 501 that includes one or more PUM 502 or one or more stakeholders 503 monitored by one or more sensors, devices, or systems (generally referred to as sensors 504) may generate SEE data 505, patterns 506, or contextually categorized/classified patterns/data sets 507 in any arrangement which may be communicated to one or more state management systems 508. The state management systems may interact with one or more tokenized care languages 509, repositories 510, or specialized care language models 511 to create, at least in part, a representation of the state of the SEE 501 or elements thereof, including one or more PUM 502, stakeholders 503, sensors 504, SEE data 505, patterns 506, or contextually categorized/classified patterns/data sets 507 including, for example, in the form of one or more tokenized care languages 509.

In some embodiments, input datasets may be processed and interpreted more accurately, faster, more effectively, and with more efficient use of resources if contextual information is applied and taken into account along with input datasets. Context may play a crucial role in interpreting and responding to inputs as context may allow a system to consider surrounding circumstances, previous inputs, and relevant information when interpreting inputs or identifying patterns.

Contextual information may include one or more data sets, patterns, pattern frameworks, event frameworks or other frameworks, specifications, calibrations or configurations, circumstances, history, or other relevant information about the PUM, the SEE, stakeholders such as caregivers, or external elements, such as weather, local events, legal context, or other factors. For example, during heat waves, a system may interpret sensor input that indicates behavioral changes in the PUM, such as fewer hours of sleep or slightly higher blood pressure as an increased risk of a heat-related critical health event.

One or more mechanisms may be used and combined in order to consider context when processing input datasets. In some embodiments, context management mechanisms may be available with AI/ML systems, where various techniques may be used to capture, retain, and use contextual information across different modalities enabling more nuanced and accurate responses to inputs. Mechanisms such as transformers, which use self-attention mechanisms to assign different weights to input tokens, allow a model to consider relevant context, including past input datasets.

In some other embodiments, context management may be done by using domain-specific training, where models are pre-trained on large datasets to learn general features and context, then fine-tuned with additional training using domain-specific datasets or through mechanisms such as transfer learning. This may result in models that are suited to specific domains or contexts. Combinations of these specialized models may be selected, based on their specific contexts, to interpret or process inputs.

In some embodiments, inputs and contextual information may be used to select a most appropriate module to interpret or process the inputs. This selection may be accomplished by various mechanisms, such as using ML-based classifiers or applying maps, rules, patterns, or pre-defined flows or relationships. This may be applied at one or more levels and may be performed on raw data sets coming from sensors, devices, or systems, events or patterns coming from pre-interpretation of such raw data allowing for context-based processing at multiple levels and locations within one or more SEE.

The use of categorizations of the one or more data sets generated by the one or more sensors, devices, or systems of a SEE may enable the data sets origins of those data sets to have a relationship with these categorizations. For example, a sensor data set may form part of a temporal category and a spatial category. In some embodiments, each of these sensors, devices, or systems data sets may contribute to a categorization where that contribution may include the data from the sensor, an identity of the sensor, or one or more weightings of that data. These data sets may form one or more patterns which in turn form, in part or in whole, one or more categories. For example, this weighting may be, in part, determined by a relative importance or value of a contribution to a category. This importance or value may, at least in part, be determined by the pattern of which such a data set is a part.

In some embodiments, each of the categorizations may be evaluated individually or collectively and as such one or more weightings may be calculated, bound, or assigned to each of these categories.

In some embodiments, there may be default values for weightings of the one or more patterns or combinations thereof. For example, a pattern forming part of a mobility event which is categorized as a spatial categorization may have a high weighting value as the PUM movement may be, for example, tracked by a set of sensors including but not limited to visual, audio, haptic, radar, or other sensors capable of tracking movement such that the pattern has a high internal consistency. Each of the sensor data sets in aggregate may be determined, for example by one or more pattern recognition systems such as a care hub, care processing system, or an edge processing system in any arrangement, to be consistent with the movement of the PUM from one location to another in a SEE.

In some embodiments, there may be default values for the one or more data sets or patterns that form a pattern framework where, for example, a mobility pattern framework has a set of N patterns which in this example is a closed set, and each of the data sets or patterns in this set has a default value, representing a relative priority of these data sets or patterns that from such a pattern framework.

In some embodiments, an action or event of a PUM may be represented by relative values of the one or more categorizations of the data sets or patterns generated by the one or more sensors, devices, or systems of a SEE. For example, an event may include spatial, temporal, behavioral, or health and wellness categorizations where the one or more data sets or patterns generated by the one or more sensors, devices, or systems form part of these categorizations with differing priority, weightings, or other relative values. Some of these relationships may have default values that are part of the pattern frameworks or may have specified weightings for the one or more categories that they are part of.

In some embodiments, the determination of these weightings may, at least in part, be undertaken by one or more AI/ML modules, where training data for these modules is based, in part or in whole, on the data sets of the one or more sensors, devices, or systems of one or more SEE in which one or more PUM is domiciled.

One aspect of the employment of AI/ML modules may be evaluation or configuration of one or more relationships between and amongst the one or more sensors, devices, or systems, the data sets or patterns generated thereby, the one or more categorizations or classifications, or the events, actions, or other activities of one or more PUM in one or more SEE.

In some embodiments, one or more repository may be used for management or storage of the one or more relationships between the one or more sensors, devices, or systems present in a SEE, the data sets or patterns they generate, or the one or more categorizations or classifications to which they have one or more relationships. In some embodiments such repositories may be in the form of one or more graph databases.

In some embodiments one or more AI/ML modules may be employed to evaluate, manage, classify, identity, or in other manners operate upon a repository and the data stored therein. This may include, for example, use of fuzzy logic and other similar techniques to identify or classify relationships between the elements stored therein. This may include identification or classification of relationships that have not been previously instantiated.

Care hubs or care processing systems may employ one or more AI/ML systems to, in part or in whole, identify a composition of elements in one or more relationships between, for example, one or more sensors, devices, or systems, data sets or patterns generated thereby, classification and categorizations of these data sets, patterns or pattern frameworks in any arrangement, one or more SEE, or one or more PUM.

In some embodiments these relationships may be static or dynamic. For example, a dynamic relationship may be between a worn or carried device (e.g. a pendant or smart phone of a PUM) that is present in a SEE, and the one or more sensors, devices, or systems that are embedded at fixed locations within a SEE.

In some embodiments, evaluation of data sets, patterns, categorizations, or elements thereof may include the use of generative adversarial networks (GAN) networks to, at least in part, determine an optimized balance between the categorizations, patterns, or elements thereof. Generally, a GAN uses two neural networks in an adversarial manner in a zero-sum game, however in addition to this approach there may be two or more neural networks which are configured to act as players in game theory-based games.

In some embodiments relationships between one or more sensors, devices, or systems and the data sets or patterns generated by them may be evaluated, for example, by one or more care hubs or care processing systems to establish dependencies of such relationships. This may be used, in some embodiments, to determine a validity or accuracy of such data sets or patterns to detect any faults with the sensors, devices, or systems.

In some embodiments, one or more sensors, devices, or systems including, for example, care hubs or care processing systems, may have available resources, including but not limited to processing power, storage, communication bandwidth, or other resources. These resources may generally be understood to form constraints on an operating performance of any technology and, in the case of AI/ML operations, may include a time, heat, processing power, training data set, or other factors.

The sensors, devices, or systems deployed or present in a SEE that are employed for the monitoring of a PUM may all have some degree of constraint, such as batteries or power sources, communication capabilities including bandwidth, processing capability, memory or other storage, or other constraining resources.

In situations where resources have limitations, one or more AI/ML model may be used identify which data sets or patterns represent, at a current or predicted time, a most likely resource to represent a health or wellness impact on a PUM. This determination can, in some embodiments, be used to direct those sensors, devices, or systems to generate data sets or patterns that involve the use of those resources of those sensors, devices, or systems to consume those resources to produce data sets or patterns of sufficient fidelity, granularity, or timeliness that may be, at least in part, used to monitor or predict a health or wellness impact on a PUM. This use of the one or more AI/ML modules or one or more care hubs or care processing systems may enable the SEE, in whole or in part, to focus on one or more PUM activities, including behaviors, and may, in some embodiments, be controlled by such AI/ML modules, care hubs, care processing systems, or other configured systems, possibly with human intervention, to focus the attention of such SEE.

In some embodiments, an attention dashboard function may be presented to one or more humans monitoring a PUM in a SEE. This may include providing a supervisory capability to one or more health professionals, for example, when a fall or other detrimental effect is detected. In some embodiments, such attention management may involve communication of this attention or focus to one or more human, machine, or combination in any arrangement.

One aspect of this approach may be use of, for example, pattern edge detections, including those with static or dynamic thresholds where, for example, one or more systems, including those employing one or more AI/ML modules, may be configured to identify a rate of change, step functions, vectors, topological features, or other metrics that indicate, at least in part, transitions from one state to another.

In some embodiments, a care hub or care processing system may include an attention/focus module that in turn may include one or more AI/ML modules that operate on the one or more data sets or patterns generated by the one or more sensors, devices, or systems of a SEE. This may include the use of one or more digital twins to evaluate or predict the operation of such attention/focus systems, for example, to evaluate an impact on the sensors, devices, or systems and their use including but not limited to consumption of resources.

In some embodiments one or more metrics for attention or focus may be employed to, at least in part, represent a state of the sensors, devices, or systems deployed or present in a SEE.

Context management may also be employed as part of an attention or focus process such that the data sets and or patterns generated by the one or more sensors, devices, or systems of a SEE include contextual information such as relationships of the PUM to other stakeholders, the PUM's contextual behaviors, including patterns thereof, or other relationships that a PUM may have.

For example, an attention or focus process may be initiated based on differences in one or more patterns that may form part of one or more categorizations such as spatial, temporal, behavioral, or wellness and health, where these pattern differences may include, at least in part, contextual data on, about, or involving a PUM. In some embodiments, such contextual data may be employed to augment input data sets, for example those generated by the one or more sensors, devices, or systems of a SEE.

In some embodiments, one or more AI/ML modules, physics engines, or digital twins may be employed to, at least in part, determine which data sets, patterns, or behaviors are prioritized for various levels of attention or focus. This may include the use of digital twins to evaluate a potential for such attention and or focus.

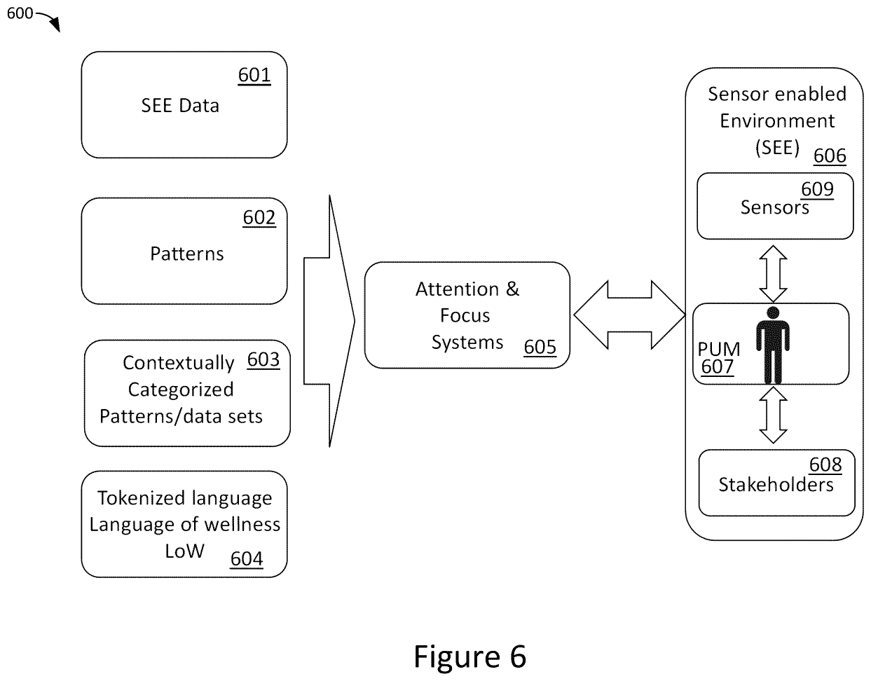

FIG. 6 illustrates a block diagram of an attention and focus dataflow 600, according to example embodiments of the present disclosure. For example, SEE data 601, patterns 602, contextually categorized/classified patterns/data sets 603, or tokenized one or more care languages, for example language of wellness (LoW) 604 may be communicated, in whole or in part, to one or more attention and focus systems 605. Such system may interact with a SEE 606, including with sensors, devices, or systems 609, one or more PUM 607, or one or more stakeholders 608 in any arrangement.

In some embodiments, a care hub or care processing system may include a response module which may communicate with one or more other systems elements. The response module may generate candidate responses to current or predicted states, events, or actions of a PUM in a SEE. This may include response candidates that may calibrate or configure the one or more sensors, devices, or systems that are deployed or present in a SEE. These response candidates may include data sets, pattern frameworks, patterns, state, configurations, calibrations, one or more languages, or other elements in any arrangement.

In some embodiments, this may include the use of one or more tokenized language models, for example a monitoring language such as language of wellness (LoW) which may be expressed as set of tokens. For example, a set of X tokens comprising token A/B/C may equate to a monitoring situation. Further languages may be employed, such as quality of life (QoL) or response candidate (RC), which may also be tokenized representations of actual or potential states, actions, events, or situations of one or more PUM in a SEE.

In some embodiments one or more AI/ML modules may contribute to determining one or more response candidates that include characteristics, including metrics of one or more languages representing the quality of life (QoL) of the PUM in a SEE. In this manner such modules may operate to evaluate a potential of response candidates to impact the quality of life of a PUM.

For example, AI/ML modules employing techniques such as retrieval augmented generation (RAG) may be employed to, at least in part, determine potential response candidates which may include use of digital twins or physics engines with which to assess or evaluate one or more contextual behaviors of a PUM in a SEE.

In some embodiments there may be one or more response frameworks which represent particular responses to likely, well known, or predicted events affecting a PUM. For example, there may be one or more fall response frameworks which may be instantiated depending on a severity of a fall as detected by the one or more sensors, devices, or systems of a SEE, which for example may be represented by one or more patterns or states. These response frameworks may be stored in one or more repositories and may be made available to multiple PUM domiciled in multiple SEE. For example, there may be a response candidate that is configured for immediate response, such as calling 911 or other emergency services. There also may be one or more standardized response frameworks, including standardized responses for known PUM adverse situations, such as falls.

In some embodiments, response frameworks may comprise one or more data sets generated by one or more sensors, devices, or systems deployed or present in a SEE, patterns of such data sets, state data, one or more categorized data sets, including spatial, temporal, contextual behavior, or wellness or other data, including calibration or configuration data in any arrangement.

In some embodiments, one or more pattern frameworks including, at least in part, data sets or patterns generated by the one or more sensors, devices, or systems of a SEE may be aligned with event pattern frameworks representing a potential health and wellness event that may impact a PUM. Such an alignment may be stored in one or more repository where one or more AI/ML modules may determine, at least in part and using, for example, one or more digital twins or one or more physics engines, likely correlations or alignments of the pattern frameworks and event frameworks as each of these is populated by the data sets or patterns generated by the one or more sensors, devices, or systems of the SEE. This determination may include the use of game theory where, for example, one or more strategies may be employed or identified to establish alignment, correlation, or causation. These determinations may, in some embodiments, be stored in one or more repositories.

In some embodiments, determinations may form one or more response frameworks where, upon population of the one or more pattern frameworks or event frameworks with the data sets or pattern generated by the one or more sensors, devices, or systems of a SEE, one or more thresholds, metrics, or other variables may be satisfied such that the care hubs or care processing systems may initiate one or more response candidates based at least in part on the one or more response frameworks.

In some embodiments, one or more care hubs or care processing systems may include the use of quality of life (QoL) metrics which may include impact analysis for selection or deployment of one or more response candidates.

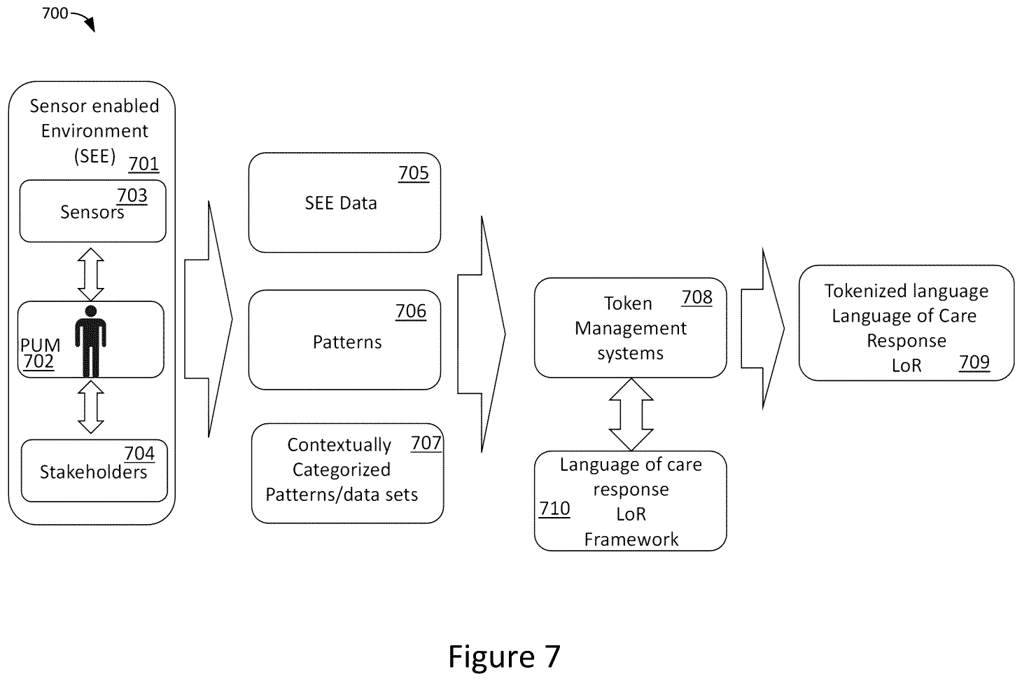

FIG. 7 illustrates a block diagram of a token management dataflow 700 for responses, according to example embodiments of the present disclosure. For example, a SEE 701 including one or more PUM 702, one or more stakeholders 704, or one or more sensors, devices, or systems 703 employed for monitoring in the SEE 701 may generate SEE data 705, patterns 706, or contextually categorized/classified patterns/data sets 707 which are communicated to one or more token management systems 708 which may employ a care language framework, for example a language of care response (LoR) framework 710 may generate a tokenized care language, for example a language of care response (LoR) 709.

Using a combination of an SEE including embedded or present sensors or devices, fixed or mobile, in combination with one or more physics engines configured to represent measurements and data sets generated by such sensors or devices, a remote monitoring capability may be instantiated using, for example, one or more virtual reality capabilities, where the SEE and the PUM therein may be visualized and represented within such a virtual environment. In some embodiments this may include one or more representations of a PUM, for example as a digital twin. The use of such digital twin of a PUM may include actual or predicted behaviors, where such PUM actual sensed behaviors may be compared, for example, with predicted behaviors based at least in part on pattern frameworks or tokenized behavior representations including one or more categorizations.

In some embodiments, such monitoring may be represented on a screen, such that a virtual PUM representation may be monitored within the SEE and the PUM's behaviors are, at least in part, evaluated by one or more AI/ML system so as to, at least in part, determine any variations that may indicate a care or wellness event.

In some embodiments, such monitoring may use, for example, cameras, microphones, haptic, or other fixed, carried, or worn sensors and devices, where the measurements and data of such sensors or devices may provide data in a format that protects privacy of the PUM, for example using tokens to represent such data which may be used to configure one or more representations of the PUM in one or more virtual environments.

In some embodiments where monitoring of the representations of the PUM deviate, vary, or in other manners indicate that a care and wellness event may be forthcoming or occurring, the monitoring may invoke raw data feeds from the SEE, for example, configuring a camera to provide live images, configuring a microphone to provide live audio, or other ways of providing live data from the SEE. Such configuration may include alerting the PUM or other stakeholders as to the change in configuration of the sensors or devices.

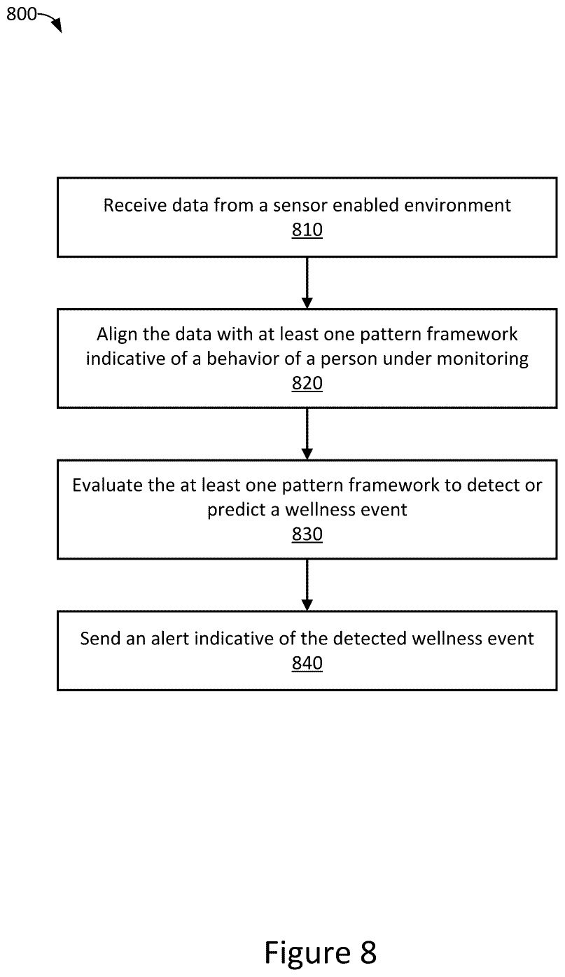

FIG. 8 illustrates a flowchart for an example method 800 for monitoring a SEE, according to example embodiments of the present disclosure. It will be appreciated that the method 800 is for illustrative purposes only, is not intended to be limiting, and is presented with a high degree of generality for ease of understanding. It will therefore also be appreciated that steps of the method 800 may themselves comprise several sub-steps, that steps of the method 800 may be excluded, and that additional steps not illustrated may be included in actual embodiments of the method 800.

At block 810, an example AI/ML model receives data from a sensor enabled environment. For example, a care hub executing a convolutional neural network trained on historical monitoring data 313 from Alzheimer's patients may receive temperature, haptic, audio, and video data from an Alzheimer's patient's home.

At block 820, the example AI/ML model aligns the data with at least one pattern framework indicative of a behavior of a person under monitoring. For example, the AI/ML model may deduce from the temperature data that a stove has been turned on in the kitchen, which may align with a cooking framework. The AI/ML model may also determine from the video and haptic data that the Alzheimer's patient has entered a bed, which may align with a sleeping framework.

At block 830, the example AI/ML model evaluates the at least one pattern framework to detect or predict a wellness event. For example, upon determining that the cooking and sleeping frameworks are active simultaneously, the AI/ML model may determine that the Alzheimer's patient has left the stove on and gone to bed, thus indicating a memory lapse event that could escalate to injury if left unaddressed.

At block 840, the example AI/ML model sends an alert indicative of the detected wellness event. For example, the AI/ML may notify one or more monitoring individuals, first responders, the Alzheimer's patient themselves, family members, or any other stakeholders.

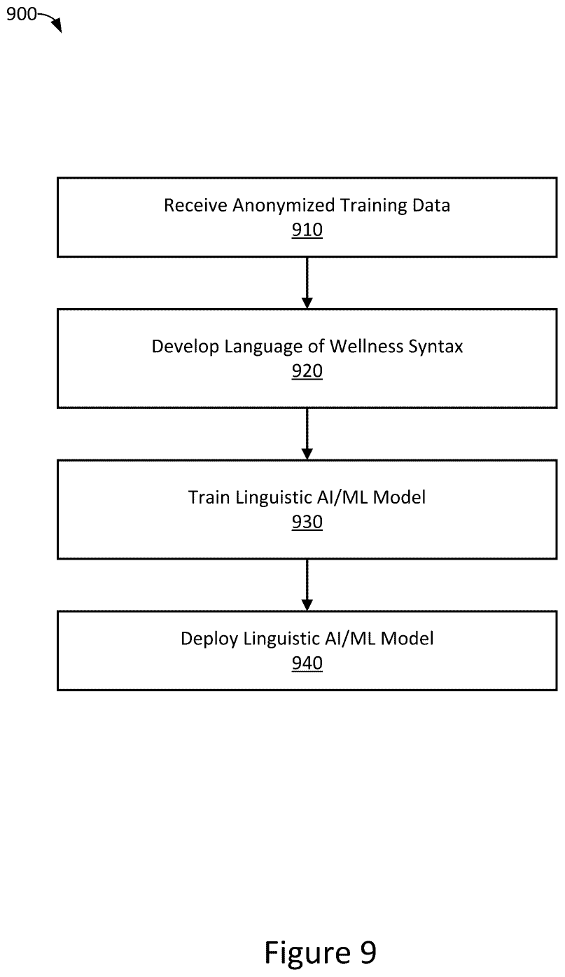

FIG. 9 is a flowchart of an example method 900 for generating and maintaining a linguistic AI/ML model for machine learning for aggregating and evaluating data from a sensor enabled environment, according to embodiments of the present disclosure.

In various embodiments, the linguistic AI/ML model generated and maintained per method 900 may be provided as a “hub” AI model for use in particular setting (e.g., a first SEE for monitoring a first PUM) after being trained initially on data not related to that particular setting. Accordingly, a linguistic AI/ML model may act as a first stage before localization or calibration tunes the model for the particular setting, or may be used in a generalized setting where the environment may be in flux, or the identify of the PUM may be in flux. For example, a linguistic AI/ML model may be used in a short-term care facility, where insufficient data may be present to tailor the model to any one PUM during an expected stay in the facility. For example, in a newly constructed or renovated SEE, insufficient data may exist regarding the layout or capabilities of the sensors, and a linguistic AI/ML model may be able to be used without need for further calibration.

Accordingly, the inventors have found several unexpected benefits of using a linguistic AI/ML model that is normally designed for handling language processing tasks, in a novel scenario for healthcare to allow for more rapid deployment, reduced training dataset size requirements, greater privacy in personal data used in the training process, compared to conventional AI/ML models used in this field. These benefits, and others that will be recognized by those skilled in the art, can be realized by treating actions, behaviors, and events identified in the SEE as semantic elements in a language of wellness (LoW), which has its own syntax and grammatical rules. When a BHWS event disobeys one or more of these rules, either predictively, or reactively, the linguistic AI/ML model may generate and transmit an alert to one or more stakeholders to mitigate or prevent harm from such actions.

To develop the linguistic AI/ML model and the related syntax for the LoW, method 900 begins at block 910, where a central service, such as a model generation system receives anonymized training data from a plurality of SEEs related to monitoring various PUM according to various associated HCP for those PUM. In various embodiments, these data are tokenized, so that tags identifying features of the data can be read without the need to decrypt all of an associated data set (e.g., to identify data relevant to a set of criteria, such as certain health conditions, certain locations of SEEs, demographic data for a type of PUM for whom the data were gathered, etc.). In various embodiments, the data are stripped of personally identifiable information or such information remains encrypted so that when data from multiple SEEs are received, the aggregated data are anonymized and information related to a particular SEE or particular PUM cannot be determined from the aggregated data set or otherwise linked back to the particular SEE or particular PUM. In various embodiments, the tokenized data identify whether a behavioral, health, wellness or safety event occurred, whether an alert was generated for a such event, whether the alert was a false or true positive or a false or true negative, and combinations thereof.

In some embodiments, a central service periodically or in response to a behavioral, health, wellness, or safety (BHWS) event occurring (e.g., a deployed linguistic AI/ML model detecting alert conditions) receives updated information to continue improving the models with. For example, a central service can receive from the computing device, tokenized alerts of BHWS events affecting the particular PUM identified via a deployed instance of the AI/ML model and update one or more data sets with the alert and data carried therein. Which data sets are updated may be based on one or more categories of the BHWS event or classification of the PUM that are readable in the tokenized alert matching or corresponding to one or more categories for training/retraining generalized AI/ML models for use PUMs having similar categories of health conditions monitored for or belonging to a similar category of PUM. For example, a tokenized alert can indicate that the token relates to a health alert of a fall affecting a person identified as between 60-80 years old, and is added to two data sets-one for persons who have fallen, and one for persons between 60-80 years old. The encrypted data in the tokenized alert can then be anonymously aggregated with the other data in the data set (e.g., reported data from sensors in the SEE, behaviors or activities identified as occurring prior to the fall, whether the fall was a false positive or true positive, whether the sensors and AI/ML model missed identifying the fall (e.g., a false negative), whether the AI/ML model correctly predicted a fall occurring and helped mitigate or preemptively alert for a potential fall, etc.).

Accordingly, the set of training data may be received from at least SEE, and is related to BHWS events affecting at least one PUM. These PUM are each associated with at least one corresponding SEE, and each SEE corresponds to at least one PUM.

In some embodiments, the central service, for example, via one or more machine learning models, can align the training data with at least one pattern framework indicative of a behavior of a person under monitoring to develop the tokens of the BHWS events. For example, the AI/ML model may deduce from the temperature data that a stove has been turned on in the kitchen, which may align with a cooking framework. The AI/ML model may also determine from the video and haptic data that the Alzheimer's patient has entered a bed, which may align with a sleeping framework. In some embodiments, the training data are received pre-aligned and are provided as tokens of the various BHWS events.

At block 920, the central service develops the LOW syntax for the linguistic AI/ML model. In various embodiments, the linguistic AI/ML model may be a large language model (LLM), retrieval augmented generation (RAG) model, a private large language model (PLLM); or a specialized language model.

As will be appreciated, linguistic models are typically designed to process natural human language, and are trained on text input in a particular language. For example, a first model may be trained on English, a second model on Japanese, and an nth model on an nth language. The present disclosure proposes that the benefits of linguistic models be applied in a new fields and in unexpected ways by rendering actions and events rendered into a language of wellness. Accordingly, the central service develops the LOW and the associated syntax by which to evaluate the BHWS events (e.g., tokenized collections of sensor data and associated analyses) occurring the in the SEE, and thereby permits the use of linguistic models in an unexpected way, which improves and expands the underlying functionality of the computing devices on which the linguistic AI/ML model is eventually deployed.

The central service analyzes the sequence of events to develop the language of wellness (LoW). This language, in common with other languages, may have a set of expressions, a syntax, a grammar, and a set of semantics. Such syntax may be influenced by various spatial, temporal, and pattern-like behaviors, which form the semantics of the language. For example, an event of brushing teeth may be frequently seen in proximity to a sleep event (e.g., on waking or prior to going to bed), but only after a meal event (e.g., not within X minutes before a meal). These rules may be set manually, or identified organically by the central service based on the way the “words” or “phrases” of the various events are put together.

In various embodiments, the syntax may be temporal, so that a group of events is examined in time or in a time window. For example, an event of waking that occurs at time t1, an event of eating (or skipping) breakfast at time t2, and an event of a low/high blood sugar event at time t3 can be understood as a temporally ordered sequence with a syntax from t1-t3. As will be appreciated, temporal syntax may include events occurring simultaneously or with overlapping time ranges. For example, an event of the stove being on from time t1-t4 may be understood with reference to a PUM eating breakfast from time t3-t8.

In various embodiments, the syntax may be spatial, so that a group of events is examined in space or in a spatial region. For example, an event occurring in a bedroom can be understood with respect to an event occurring in a kitchen (e.g., a first PUM waking while a second PUM cooks breakfast). For example, an event of a fall occurring may be understood with a spatial reference to a bathroom (e.g., slipping on a wet floor) differently than a fall occurring in a living area (e.g., tripping on a loose carpet) and differently than a fall occurring in a sleeping area (e.g., falling out of bed).

In various embodiments, the syntax may be relational, so that a group of events is examined with respect to the identity or types of events in that group. For example, events of teeth brushing and showering may be identified as being syntactically related, as are events of showering and falling, whereas events of teeth brushing and a falling are not.

As will be appreciated, the syntax may include various combinations of different temporal, spatial, and relational constructions and interrelationships. These relationships may be used predictively by the end user, to identify when a dangerous, medically significant, or undesirable event is expected to occur and potentially ameliorate or prophylactically avoid the event occurring. For example, if the syntax predicts that a PUM having skipped breakfast is at risk for a low blood sugar event, the linguistic AI/ML model may produce a predictive alert for the PUM to eat something sugary to avoid the low blood sugar event.

These relationships may also be used reactively by the end user, to identify when an event has occurred that deviates from an expected syntax, but may constitute a dangerous, medically significant, or undesirable event. For example, when the syntax indicates that the PUM should perform action 1, action 2, or action 3 after a particular series of BHWS events is observed, but a third action occurs, the linguistic AI/ML model may produce a reactive alert for the PUM. For example, a PUM who leaves a bedroom in the morning and enters a kitchen, may be expected to remain in the kitchen (e.g., to cook and eat), return to the bedroom (e.g., to retrieve a forgotten item), or head to a bathroom (e.g., for morning toilet). However, if the PUM repeatedly leaves and returns to the bedroom, such as during a Alzheimer's event, the AI/ML model may identify that the repetitive behavior is unusual and disobeys an syntactical rule developed in the syntax used by the AI/ML model, and therefore a reactive alert is generated.

At block 930, once the LOW and the syntax is developed, the central service trains the linguistic AI/ML model using the rules of this language, as one of skill in the art would understand to train a typical linguistic AI/ML model. As will be appreciated, linguistic AI/ML models operate on prompts and produce outputs according to the corpus on which they were trained and the prompt. For example, by developing the LoW, the central service can provide a model that accepts as part of the prompt one or more of: the tokens of events occurring in the SEE, raw (encrypted or unencrypted) sensor data from the SEE, details of the PUM or SEE, lists of events occurring in the SEE or affecting the PUM, and the like.

At block 940, the central service deploys the AI/ML model. In various embodiments, the central service hosts the AI/ML model as a “hub” AI that the computing devices located (remotely from the central service) transmit prompts to, so that predicted or analyzed results are returned to the requesting computing device without having to host an instance of the linguistic AI/ML model itself or transmit large amounts of sensitive data for detailed analysis on a remote system. Accordingly, the linguist AI/ML model acting as a hub can improve data security and reduce network traffic, among other benefits, while offering high-quality analysis of the PUM in various locations.

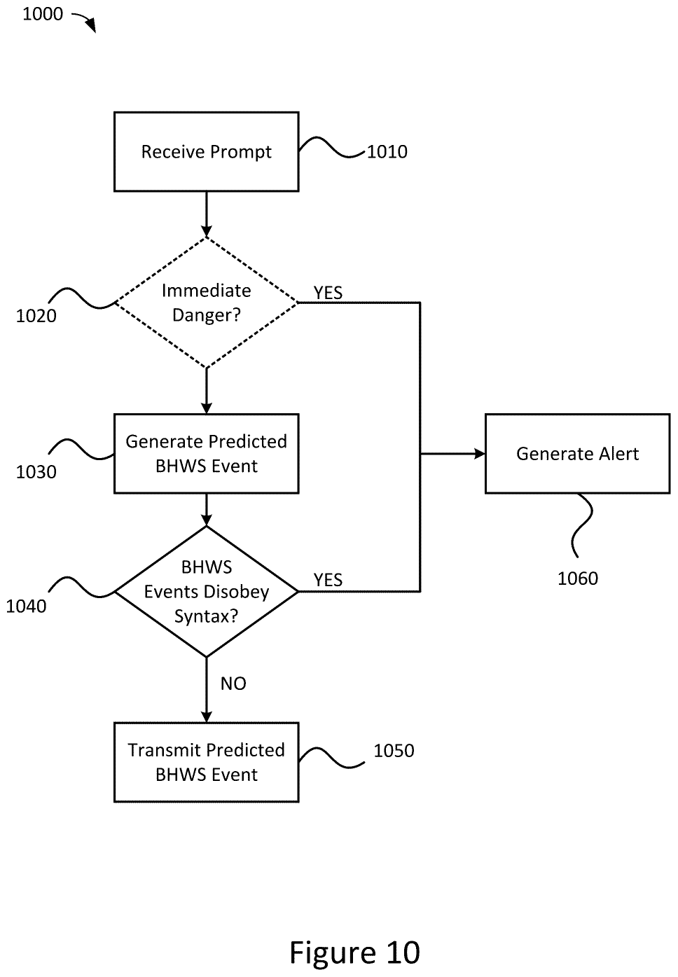

FIG. 10 is a flowchart of an example method 1000 for deploying and using a linguistic AI/ML model for machine learning for aggregating and evaluating data from a sensor enabled environment. Although generally discussed in the context of deploying the linguistic AI/ML model in a hub AI configuration, the present disclosure contemplates that instances of the “hub” linguistic AI/ML model may be deployed to various computing devices for localized use as “edge” AI models or across multiple platforms as “mirrors” of the hub linguistic AI/ML model. In various embodiments, the deployed linguistic AI/ML model may be the model generated according to method 900, which is configured to process prompts according to a language of wellness (LoW) syntax using one of various formats of language-based AI/ML models (e.g., RAG, LLM, PLLM).

At block 1010, the linguistic AI/ML model receives a prompt. In various embodiments, the prompt is received from a computing device associated with a particular SEE (and one or more particular PUM), and which may be located remotely from the server or computing device on which the AI/ML model is deployed, and may include one or more BHWS events.

In various embodiments, the presently described linguistic AI/ML model allows for greater data security, as tokenized behaviors included in the prompts can remain encrypted and anonymized, and constitute a smaller amount of data needed to be transmitted for analysis than in conventional systems.

The present disclosure contemplates that a BHWS can describe various incidents that may be classified as one or more than one of a behavioral incident, a health incident, a wellness incident, or a safety incident for various PUM, and may be determined by interactions among several such incidents or events to describe an emergent event. Additionally, BHWS incidents may include both “positive” and “negative” incidents, or incidents that be characterized in multiple ways or as multiple ones of behavioral incidents, health incidents, wellness incidents, or safety incidents. For example, a dementia flare-up may be classified as both a health event and a behavioral event, and may include several other BHWS events that, when combined, describe the BHWS event as a dementia flare-up event in addition to or instead of as the individual events thereof. Additionally or alternatively, as an inverse to a dementia flare-up event, a lucidity-break (e.g., a positive vs. negative event) may be monitored and alerted for using the same or different sensors and the same or different alerting conditions.

For example, a first PUM may be monitored for the presence of the behavior of “eating breakfast”, while a second PUM may be monitored for eh absence of the behavior (e.g., “skipping breakfast”), which may be classified as a behavior incident as well as a health incident, wellness incident, or a safety incident depending on the HCP for the PUM, and the occurrence or timing of the BWHS incident may be treated positively or negatively according to the HCP. For example, both the first and second PUM may need to avoid eating breakfast due to medications needing to be taken on an empty stomach, so “eating breakfast” may be handled as a negative incident (e.g., resulting in an alert), whereas “skipping breakfast” may be handled as a positive incident (e.g., not resulting in an alert). In a contrasting example, both the first and second PUM may need eat breakfast due to blood sugar requirements, so “skipping breakfast” may be handled as a negative incident (e.g., resulting in an alert), whereas “eating breakfast” may be handled as a positive incident (e.g., not resulting in an alert).

In various embodiments, BWHS incidents (or events) may be triggered via detection of a one-time or an ongoing condition in the SEE or affecting the PUM. For example, a BWHS incident may monitor whether a PUM has fallen, and is indicated in response to a sound, impact, positional sensor, or combination thereof indicating that the PUM has fallen. In another example, a BWHS incident may monitor whether the PUM is affected by a tachycardia condition, and is indicated in response to a heart rate monitor indicating a heart rate above a threshold rate for at least a threshold time (e.g., to avoid false positives from day-to-day excitements).

In various embodiments, the prompt may include a class or other details of the PUM or a healthcare plan (HCP) for the PUM, which may include various non-personally identifiable information relevant to the health of the PUM. For example, the prompt may identify an age, gender, reason for monitoring, available sensors associated with the PUM, available sensors available in the SEE, etc. In various embodiments, the linguistic AI/ML model and the requestor may agree on using a unique identifier so that multiple prompts can be linked over a period of time for longitudinal analysis of the PUM or SEE, without linking the results in an identifiable way to third parties with the PUM or the SEE.

In various embodiments, the prompt may include one or more tokenized observations of the SEE or PUM. In various embodiments, the tokenized observations may omit some or all of the underlying sensor data used to reach the identification of a particular event included in the prompt. For example, rather than sending the raw (encrypted) data that lead to a determination that a PUM suffered a fall event at time T at location L, the tokenized observation may indicate that a fall occurred at time T at location L with no further data attached. In an additional example, the tokenized observation may indicate that a PUM suffered a fall event at time T at location L, and include some or all of the sensor data to provide additionally context for syntactical analysis of the event (or surrounding events). In various embodiments, the linguistic AI/ML model may request that a prompt include or exclude certain data and query the requestor for such data if the data are desired.

At block 1020, the linguistic AI/ML model optionally determines whether an immediate danger state is identified in the BHWS included in the prompt. In various embodiments, an immediate danger state is identified based on a current state of the PUM or SEE being associated with a currently detected condition identified with an alert condition. For example, temperature of over X degrees Fahrenheit in the SEE may be generally present to indicate a fire in the SEE, which, when detected, is classified as an immediate danger state that causes an alert to be generated. In an example, detection of an open window may be classified as an immediate danger state for a PUM with dementia (e.g., for an increased risk of exit from the SEE) if the prompt indicates that the PUM is a dementia patient, but not if the prompt indicates the PUM is otherwise healthy. In an example, detection of a PUM on the floor of the SEE for at least X minutes be classified as an immediate danger state (e.g., indicative of a fall or other BHWS event) unless the prompt indicates that the PUM frequently engages in activities that occur while lying on the floor (e.g., yoga, stretching, prescribed/preferred time lying on a hard surface, etc.), and the linguistic AI/ML model may ignore such data as not indicative of an immediate danger state in the particular case.

When the computing device that transmitted the prompt indicates that such immediate danger states are handled locally (e.g., via a watchdog application), the linguistic AI/ML model may omit performing block 1020, or may include any alert generated per block 1060 in a transmission to the prompting computing device to handle locally. When an immediate danger condition is detected, method 1000 proceeds to block 1060 in addition or alternatively to proceeding to block 1030.

At block 1030, the AI/ML model generates one or more predicted BHWS events based on the prompt. In various embodiments, the provision of current and historical BHWS events allows the AI/ML model to predict potential future BHWS events using the syntax of the LoW. In various embodiments, the linguistic AI/ML model identifies the current state of the particular PUM and the particular SEE based on the prompt, and identifies one or more digital twins of the PUM, the SEE, or other entities in the SEE to simulate what the twinned entity will do next to generate the predicted BHWS event.

In various embodiments, each digital twin incorporates specifications of the capabilities of the entity that the digital twin is associated with. Each digital twin incorporates the physical characteristics of the entity and represents the state of the entity within a simulation of the environment. The interactions between digital twins provide an accurate and timely predictive representation of the interactions between the entities, which can be used to generate candidate next states over a plurality of iterations, where more-likely candidate next states are simulated more often than less-likely candidate next states.

In various embodiments, digital twins can represent the care and wellness state of a PUM and the environment with sufficient fidelity so as to be used in predictive analytics, including the use of machine learning, for the care and wellness benefit of the PUM. In many circumstances the digital twin can be one of a set of digital twins representing a set of PUM that have a common set of care and wellness characteristics, such as the same or similar HCP and the operating patterns and pattern elements thereof. Accordingly, the digital twin can comprise a dynamic tokenized representation of quiescent or active behaviors of a specific PUM in a specific environment or represent a generalized model of a PUM in a generalized environment, which may be localized or used as-is. The tokenized behaviors can identify or name the observed behavior, thereby labeling the token (of Bevoken) as corresponding to a specifically identified behavior, and keeping the data used to reach that identification encrypted.

The degree of disclosure of the data sets pertaining to a PUM to a digital twin may be sufficient for the Digital Twin and any associated analytic or predictive processing to be able to undertake effective predictive, trend, underlying care framework identification or other care and wellness benefit processing. This can be achieved in a number of ways, employing differing embodiments, for example the tokens may include a set of specifications that can be interpreted by a suitably authorized digital twin that can access, potentially on a time or function limited basis the data that are deemed private by a PUM for a specified purpose. This type of disclosure may be agreed by a PUM in advance. The data received by the digital twin may be expunged after the appropriate analytics or processing has been undertaken. In some embodiments, the digital twin may operate as a proxy for a PUM, such that all data are available to a digital twin, and the digital twin acts as the privacy guardian of the PUM, enacting and enforcing the privacy choices of the PUM. In a further embodiment, there may be tokens that are digital twin specific and include further specifications determining the use, propagation, or configuration of the tokens and the digital twin operating upon them. Accordingly, the digital twin, through one or more configurations, retains a trust relationship with the PUM, such that the data a PUM deems private remain private, and the digital twin can function to support the care, wellness or safety monitoring of the PUM to the benefit of the PUM.

In various embodiments, a plurality of digital twins can be used to simulate a single PUM with different configurations or monitored behavioral, health, wellness or safety (BHWS) conditions so that each digital twin can produce different predictive results. For example, a first digital twin may be configured to monitor for the PUM falling, and is configured to represent the PUM in a distracted state (e.g., based on movement patterns trained on sleepy, feverish, or inattentive historical users), a second digital twin may be configured to monitor for the PUM falling, and is configured to represent the PUM in an alert state (e.g., based on movement patterns trained on rested, healthy, or assisted historical users), and a third digital twin may be configured to monitor the PUM for heart attacks (e.g., based on historical conditions of users when struck with a cardiac event).

In various embodiments, the digital twins may use, or be used in conjunction with, one or more game theory models to, at least in part, identify potential behaviors of the PUM expressed as data sets representing patterns or to rank those behavior sets into one or more ordered arrangements. In various embodiments, wherein the plurality of candidate next states can be analyzed as a Markov chain from the current state as contextual behaviors depending from the current state with the digital twins affecting the weightings of the next states in the chain from a current state.

At block 1040, the AI/ML model determines whether the BHWS events included in the prompt, received historically that can be linked to the prompt, or generated as predicted BHWS events disobey one or more rules of the syntax of the LoW.

In various embodiments, disobeying the one or more rules of the LoW syntax includes disobeying an established spatial zone in the particular SEE, as a spatial rule violation, wherein the predicted BHWS is predicted to occur outside of the established spatial zone. For example, a PUM moving repeatedly between locations, or performing a tasks in a wrong location (e.g., washing dishes in a bathroom sink vs. a kitchen sink) may be identified a spatial rule violations that may prompt a syntax violation indicative of a dementia event.

In various embodiments, disobeying the one or more rules of the LoW syntax includes disobeying a time window, as a temporal rule violation, wherein the predicted BHWS is predicted to occur outside of the time window, wherein the time window is one of an absolute time window during a day or a relative time window from performance of a previous behavior by the PUM. For example, a PUM dressing to leave the SEE at 3 in the morning may be identified as a temporal rule violation for a potential escape or unauthorized activity event.

In various embodiments, disobeying the one or more rules of the LoW syntax includes disobeying an established order for performing a first behavior relative to a second behavior, as a pattern rule violation. For example, a PUM dressing (e.g., a dressing event) before taking a shower (e.g., a bathing event) may be identified as a pattern rule violation indicative of a potential dementia event.

In various embodiments, disobeying the one or more rules of the LoW syntax includes disobeying a combination of spatial, temporal, or pattern rule violations. For example, a PUM taking a shower and not getting dressed at least X minutes after the bathing event concludes may be identified as one or both of a rule violation for a temporal rule and a pattern rule.

In various embodiments, the determination that a rule violation has occurred may be classified as predictive (e.g., based on the predicted BHWS event causing the violation), reactive (e.g., based on the BHWS events in the prompt causing the violation), or both.

At block 1050, the AI/ML model transmits the predicted BHWS event to the computing device form which the prompt was received, which may locally determine whether the PUM complies with or deviates from the predicted BHWS and generate various alerts, wellbeing checks, or other actions accordingly. In various embodiments, the local determinations are made based on data deemed too sensitive to transmit or store outside of a network environment for the SEE (e.g., a healthcare plan), which allows for the robust provision of AI tools via a centralized hub, while still preserving privacy of the end users.

At block 1060, the AI/ML model generates an alert. In various embodiments, the alert may be transmitted from the central service to the SEE to address an ongoing or predicted BHWS event, with various amounts of encryption or tokenization applied thereto to maintain data privacy and reduce network load, while still addressing the underlying health concerns. Additionally, one or more alerts may be generated simultaneously or in sequence to one another when block 1060 is performed. The alerts may include immediate danger alerts or syntax deviation alerts (predictive or reactive) depending on the circumstances in which the alert is generated (e.g., according to determinations made according to block 1020 and block 1040, respectively), and combinations thereof.

For example, when messaging to the SEE, the alert may be directed to the PUM or a stakeholder to determine whether an identified BHWS event actually occurred, and generate a response, which can include requesting permission for various follow up actions. For example, when monitoring a PUM for fall risk, a microphone detecting a loud sound and a positional sensor identifying that the PUM is in a prone position may result in a detection that the PUM has fallen. The AI/ML model may generate an alert for transmission to the SEE for a stakeholder or other caretaker present in the SEE to check on the PUM, for the PUM to self-report a status, etc. A responder to the alert in the SEE may indicate that a fall actually occurred and may positively authorize the AI/ML model in a reply to the alert to place an alert with an outside party (e.g., an ambulance service, emergency medical service provider, or non-emergency healthcare provider), indicate that a fall actually occurred and deny authorization for the AI/ML model to place an alert with an outside party, or indicate that no fall occurred (e.g., a false positive for a fall was detected).

For example, when messaging externally to the SEE, the alert may be directed to a stakeholder who has been preapproved or in indicated in the prompt for receiving certain classes of messages in certain situations. For example, a stakeholder of a relative may be contacted with an alert under condition set one, while emergency medical services may be contacted with an alert under condition set two.

In various embodiments, the external alerts are generated as tokens, which include various data sets that are encrypted, but are useable by recipients in an encrypted or partially decrypted form, and one token may include data encrypted for the exclusive use by some recipients but not others of a particular alert. For example, if an alert is generated in response to detecting that the PUM has fallen for transmission to a sets of three stakeholders of a primary care physician for the PUM, to a stakeholder of an ambulance service, and a stakeholder of a family member, the token may indicate to all three (in an unencrypted or partially decrypted state) that the PUM has suffered a fall. The alert may include data related to the lead-up to the fall and the behaviors and biometric information useful to the primary care physician, which may be of limited interest to the ambulance service or the family member (and of interest to the PUM to keep private). Similarly, the data unencryptable by the ambulance service may include address information and keycodes necessary to access the SEE (e.g., gate codes, security alarm codes, etc.) that are of limited interest to the primary care physician or the family member (and of interest to the PUM to keep private). Each stakeholder may receive the token and use, in a decrypted state, the portions that are relevant to their interested in monitoring and treating the PUM without accessing data not necessary or deemed private by the PUM for the alert-worthy situation.

Various data sets or functional models may be provided between different parties as tokens, which act to encrypt various portions of the data. For example, a token can comprise a detected data set representing behaviors of a PUM in an environment, wherein the token is encrypted using an encryption key. The tokens may contain the sensor data or may reference the data stored at the sensor. Other devices in the system or the server may make decisions on event response or escalation, without the need to access the information stored or referenced by the tokens-without decrypting the data, the token is deemed sufficient evidence of a determination or detection based on the data. Other devices within the system may obtain the data associated to the token and use those data to, for example, enhance the event detection accuracy or to confirm the event. For example, a device may interpret a combination of acceleration and change in altitude from sensors as a “fall” event for a PUM, and issue a token associated with the sensors' data and send that event token to the server and to a nearby edge device. While the server may trigger a notification to a call center or to smart speaker app to initiate a conversation with the PUM, the nearby edge device may use the token (and not the full set of data that resulted in the data), combined with its identification or other authorization key, to request the event data from the device and use it to confirm or add accuracy to the fall event, by combining the sensor data with data from its own sensors, for example audio signals from a microphone or microphone array, or output signals from one or more Frequency-Modulated Continuous-Wave (FMCW) radar sensors.

In some embodiments, the encryption key is selected based in part on the detected data set, the at least one stakeholder, on the person under care, a type of event detected by the environmental sensor, or is unique to a session of the person under care.

One aspect of the use of LLM/LCM and other AI/ML systems with data sets generated by one or more sensors, devices or systems, for example those deployed in a SEE, including data sets that comprise streams of contiguous data, is the segmentation or quantization of those data sets into elements that can be processed by an LLM/LCM or other AI/ML system in an efficient and effective manner. This aspect can include using large language models (LLMs) and latent context models (LCMs) alongside AI/ML systems to process contiguous data streams from sensors, devices, or systems in a SEE. This processing includes segmenting and quantizing raw data into structured elements, enabling efficient processing by LLMs/LCMs. The process may involve identifying features such as movement patterns, acoustic signatures, or contextual metadata that are relevant to fall detection or other wellness, health or safety events. This processing can include the identification of features and characteristics within the data sets or the context of such sets.

One focus of such segmentation or quantization is the early and accurate identification of events, movements and other occurrences that have or can have an impact on the health, wellness and care of a PUM in a SEE, such that the identification of the actual or potential impact of the measurements of these events, movements or occurrences is evaluated at the earliest possible time. This evaluation can include the determination of the risk, for example, expressed in risk metrics, of the actual or potential impact of the measured event, movement or occurrence, and can include metrics that represent the short term, mid-term or long term actual or potential impact.

In some embodiments, one objective is to detect fall-related events (e.g., sudden acceleration, impact sounds) and contextual patterns that indicate risks to a Person Under Monitoring (PUM). This detecting and monitoring enables real-time risk assessment, including short-term, mid-term, or long-term impacts on the health or safety of the PUM, which can include the generation or deployment of one or more tokens or tokenized data representations. For example, a process may generate tokenized data segments, such as time-stamped audio features (e.g., frequency bands, decibel levels) or metadata markers (e.g., “fall detected at X:XX:XX”), which are optimized for efficient LLM/LCM processing and include encryption or access controls.

In some embodiments, management of the data sets generated by the one or more sensors, devices or systems of a SEE which are, at least in part, employed to monitor a PUM, can include a number of approaches. Embodiments may employ multi-sensor fusion and real-time analytics to prioritize data streams that show early signs of risk, for example, abnormal gait patterns, audio anomalies or other data sets that exceed one or more thresholds of the configured one or more sensors, devices or systems monitoring a PUM, which can include verifying sensor readings against calibrated thresholds to reduce the occurrence of false positives.

One approach is to segment the data sets into manageable entities which can be formatted for further processing by, for example, LLM/LCM, other AI/ML systems, care processing systems care hubs and the like. The way segmentation is undertaken by the one or data management systems, can include the calibration, configuration and operations of the one or more sensors, devices or systems. For example, if a sensor, device or system has been configured to identify one or more features in a data set, for example using thresholds, pattern matching, arrays, matrices, topologies or other techniques and criteria, then these features may be passed to one or more further sensors, devices or systems, including to one or more LLM/LCM, AI/ML systems, care processing or care hubs.

In one example embodiment, game-theoretic models can be integrated or deployed, where sensor data are treated as “moves” by players (e.g., sensors, LLMs, LCMs) to optimize segmentation strategies based on reducing (e.g., minimizing) false positives while increasing (e.g., maximizing) detection accuracy. For example, use of game-theoretic models can include where the potential and actual features of a data set are evaluated as moves in a game theory game where each of the one or more sensors, devices or systems operate as players in that game.

In this manner, one or more AI/ML systems including LLM/LCM or one or more specialized LLM/LCM may be invoked or deployed to process the one or more data sets, including those that have been segmented or quantized by one or more other AI/ML systems, including LLM/LCM or by other specialized systems configured to do so.

Often the nature of the data sets generated by the one or more sensors, devices or systems is such that the data sets can have an inherent format, such as patterns, behaviors or other delineated data sets that can be processed by one or more AI/ML systems, including LCM/LLM. In some embodiments, this formatting can include the use of common formats and protocols, such as Message Queuing Telemetry Transport (MQTT), “Matter”, “MCP” (Model Context Protocol) or more low-level formats such as H.264/265 and the like. Many datasets from wearables (e.g., accelerometers) and environmental sensors (e.g., microphones) already follow standard formats like MQTT or H.264, which are compatible with LLM/LCM processing without requiring additional transformation.

For example, in a SEE there may be one or more sensors, devices or systems that have been calibrated and configured to identify one or more patterns of the measurements being taken where such calibration or configuration includes such formatting. In some embodiments, this formatting or data sets delineation can include multi-dimensional data sets, patterns, behaviors, tokens and the like. Tokens can include tokenized data sets of any type and can include segmentations where differing data sets can be managed by differing specifications, such as access controls, user identities, time or other temporal constraints and the like.

In some embodiments, such data delineation, formatting or structure may not be present or may be present only in part. For example, in continuous audio streams (e.g., from microphones), contextual segmentation can be applied, where LLM/LCMs identify anomalies (e.g., sudden loud noises or other abrupt changes in stream data) by analyzing temporal patterns or cross-referencing with historical data.

In this example, the data stream may be continuous and the potential entry and exit points for any segmentation of the data set may not be part of that data, for example when the data comprises a stream, for example, a set of integers representing, for example, a measurement of one or more characteristic of a SEE by one or more sensors, devices or systems. For example, such a set of integers can be expressed in many differing variations, such as arrays, matrices, sets, polynomials or other mathematical expressions; however, the segmentation of these data into one or more formats that support the operations of one or more AI/ML systems, including an LLM/LCM, specifically an AI/ML system that operates to identify health, wellness or care conditions or events for a PUM, necessitates that the context of the data be available.

This approach where the data, features thereof or context of such data can be identified and managed, in combination or separately, including for example by one or more AI/ML systems, including LLM/LCM, enables determination by such systems of those sets of data that represent or have relevance to specific situations, events or occurrences that can have actual or potential impact on the wellness, care or health of a PUM in a SEE. For example, this approach can include integration of context-aware tokenization that enables LLM/LCMs to prioritize fall-related audio features (e.g., impact sounds, PUM calls for help and the like) over background noise and link the fall-related audio features to predefined risk thresholds.

In some embodiments, one or more data management systems may segment, arrange or classify these data sets generated by the one or more sensors, devices or systems of the SEE, using for example, features, patterns, behaviors, movements, locations, temporal measures, contextual or other specifications and can include the use of knowledge bases, RAG's, game theory engines, risk and other metrics, events and occurrences and the like and can involve any combination of sensors, devices or systems in any arrangement. For example, such data management can include segmentation that leverages multi-dimensional feature extraction, including audio spectrograms, acceleration vectors, and spatial metadata to train LLM/LCMs on fall detection models.

Separation of context from data sets can enable evaluation of the sets of overlapping or potentially overlapping contexts that represent, in part or in whole, the health, wellness and care journey that a PUM is traversing in or interacting with a SEE. For example, by separating contextual metadata (e.g., PUM's medical history) from raw sensor data, LLM/LCMs can tailor risk assessments to individual profiles and adjust one or more thresholds dynamically.

For example, the data streams from one or more sensors, devices or systems monitoring a PUM in a SEE may include context markers, which can, for example, be represented by vectors, which when passed to one or more AI/ML systems, including LLM/LCM, provide a feature, pattern, behavior or other PUM characteristic, which can be integrated into the processing of such AI/ML systems. This integration can be of particular importance when discerning whether such data are representing a predicted or actual health, wellness or care incident, such as, for example, a fall, heart attack or other potentially critical event. For example, audio data streams (e.g., from microphones) can include context markers as time-stamped vectors, such as “loud impact at location X” or “fall detected,” which are fed into LLM/LCMs for immediate alert generation.

In some embodiments, these data sets, form a data stream, for example those of a SEE, which may be segmented into sets that are suitable for processing by an LLM/LCM or other AI/ML system. This segmentation can include temporal segmentation, for example, every time-period, for example X seconds. This time-based segmentation can include the use of windowing, where overlapping time periods are used for segmentation and may include the use of one or more caches or buffers. However, this approach may not necessarily recognize the context of the data sets nor the interconnectedness of those data sets.

To address this situation, a contextual segmentation approach can be used, where, for example, if the values of the data or the output of a process applied to values of the data, such as, for example, a mathematical calculation, a neural network and the like, exceed one or more thresholds or other criteria, for example, including one or more features, then one or more markers can be bound to the data sets representing where features, thresholds breaches, including approaching such breaching, or other criteria are detected. These markers can delineate, for example, the start and end of a segment, the data in a segment that correlate to a threshold breach, feature or other criteria, and can in, some embodiments, include further data, such as metadata, for example, the identity of the originating source of the data, location, configuration or additional data of the sensors, including on the body, devices or systems and the like.

For example, for a data stream “A” that comprises, for example, data sets from one or more sensors, devices or systems, for example a microphone, camera, haptic sensor or worn sensor that includes, for example, an accelerometer, altitude detector or gyroscope and a second worn device that for example, includes a heart rate monitor, there could be a total of seven or more individual data streams that can be segmented and processed by an LLM/LCM or other AI/ML system. This processing can be on an individual stream or aggregations thereof in any arrangement. For example, of a set of seven sensors, data from sensor₁, sensor₃ and sensors may be processed by one AI/ML system, such as an LLM/LCM configured for those sensors, and the other sensor data sets may be processed by one or more other AI/ML system, including LLM/LCM arrangements.

These measurements represented by the data sets can occur over one or more time periods, for example, where each of the sensors, devices or systems have a common time-base, these measurements can be aligned to that time-base, and, as such, can form one or more patterns, which can be identified by the one or more LLM/LCM or by other AI/ML systems and other systems such as care hubs or care processing which can, in some embodiments, include further LLM/LCM or other AI/ML systems.

In some embodiments, one or more LLM/LCM may be employed to evaluate the incoming data streams and assign one or more delineation markers, including those that identify context, identifying the entry or exits points for a specific data set, effectively translating such data set into an embedded sequence of “words” that can be processed by one or more LCM/LLM.

This example, data stream “A” can occur over a time-period of Y, where, for example, none of the configured thresholds that the sensors, devices or systems or one or more care processing systems, including care hubs, have been breached or have a trend or trajectory that indicates a potential breach. This state is the quiescent state of these sensors, devices or systems that are monitoring a PUM in a SEE.

However, if one or more of the sensors, devices or systems or a care processing systems such as care hub or other monitoring service that has been configured with one or more thresholds for these data sets identifies that the data sets have exceeded a threshold or are approaching such threshold, then one or more markers can be bound to that data, including portions thereof, to indicate this change of state.

In some embodiments, such embedded sequence of “words” may comprise high dimensional vectors of the one or more data sets, which can represent the relationships between these one or more data sets, proving a context and meaning thereof. For example, such vectors can include, for example, acceleration, vertical or horizontal motion, velocity or other measured characteristics.

In some embodiments, data sets, vectors or other dimensions can, at least in part, form patterns, which is some embodiments have been previously determined by, for example one or more systems, including care processing, care hubs, one or more sensors, devices or systems that have been calibrated or configured to do so, in any arrangement and may include one or more AI/ML systems including LLM/LCM.

For example, a pattern can include the measurements of a PUM's movements in a SEE, for example rising from sitting to standing may be a pattern, which is repeated by the PUM over a period of time and can include measurements, by, for example, one or more worn or carried sensors, devices or systems, for example a PERS device, smartphone, smartwatch, fitness tracker and the like, which can include accelerometers, gyroscopes, altimeters, laser or other non-visual or visual measuring devices, audio, video or haptic capabilities or the like, which may be complimented by those sensors, devices or systems that are preset in the SEE, such as those that are embedded, for example haptic sensors, such as strain gauges, cameras, microphones, lasers and other RF sensing and the like where the data sets generated thereby form part of such pattern. In some embodiments, this pattern identification can be personalized to a particular PUM's movement patterns or condition, through machine learning techniques or comparison to recent/historical patterns of the same PUM.

These data sets and the patterns thereof, can in whole or in part be used in both supervised or unsupervised learning by one or more AI/ML systems, including LLM/LCM. This use in learning can include, for example, where one or more model is trained on these patterns. For example, in some embodiments, supervised or unsupervised learning can be employed to train one or more models, for example, environment awareness, pattern or meta-context models in any arrangement, which can include training one or more model that incorporates, at least in part, any or all of such models.

In some embodiments, the system undergoes rigorous validation using cross-modal datasets that include both audio and inertial sensor data (e.g., accelerometers, gyroscopes) to improve fall detection accuracy. Metrics such as F1-score, recall, and false alarm rate are continuously monitored during training and deployment. The LLM/LCM systems are fine-tuned for low-latency inference using quantization techniques (e.g., 8-bit integer weights), ensuring real-time performance on edge devices while maintaining robust fall detection capabilities even in noisy environments.

This binding of the state of the data sets, that are within a set of calibrated, learned or configured thresholds or exhibiting one or more trend that is likely to breach such threshold or matching a previously determined pattern, provides an effective means to monitor the state of a PUM in a SEE, where if the monitored patterns, behaviors, movements or other occurrences of the PUM are within the configured thresholds, the state of the PUM is determined to be quiescent. If these monitored patterns, behaviors, movements or other occurrences of the PUM exceed or have a trajectory to exceed such configured thresholds, the binding of the state can indicate this situation and can include descriptions of that state, if previously identified or classified, which can result in one or more actions, which can include the generation of communications, including alerts.

In some embodiments the binding of markers, state or metadata may be in the form of one or more tokens.

This contextual segmentation approach can, in some embodiments, include the use of various models representing differing granularities, perspectives or abstractions of the one or more contexts of the one or more data sets generated by the one or more sensors, devices or systems present in a SEE. In some embodiments, a system can employ three-tiered models, for example: (1) Environment awareness (e.g., room layout from cameras), (2) Pattern recognition (e.g., gait analysis from accelerometers), and (3) Meta-context (e.g., the medical conditions of a PUM), each optimized for specific LLM/LCM tasks.

For example, there can be a set of models, any or all of which can be employed, in part or in whole, by one or more monitoring systems to generate communications, including alerts that represent actual or potential events that can have an impact on the health, wellness, care or safety of a PUM. These communications can be provided to other systems, AI/ML systems, stakeholders, such as carers, medical professionals, including for example those attending an event, such as Emergency Medical Technicians (EMT), neighbors, relatives and any other authorized third parties.

In some embodiments, these models can include an Environment Awareness Model, which can include sensor, device or system data sets that represent the state of the SEE and a PUM therein. For example, an Environment Awareness Model can include sets of data from a selection of sensors, devices or systems in any arrangement. These data sets can be raw, that is in the format generated by the sensor, device or system and may include one or more markers or other delineation artifacts that indicate, at least in part state or context. For example, these data sets can include, measurements of the alignment of the body of a PUM to vertical or horizontal surfaces within a SEE, haptic or audio detection of the footfall of a PUM traversing a SEE, location and relationships of fittings and furniture of the SEE to the PUM and the like.

In some embodiments, these models can include a Pattern Model, which can include one or more known or pre-existing patterns that represent the state of the SEE or PUM therein. These patterns can be of any granularity and can include those generated by one or more sensors, devices or systems present in the SEE. These patterns can represent, for example, movements, such as of a limb or person, sitting, standing and the like or any other activity. In some embodiments, a pattern can comprise, for example, a set of features, such as those identified by the one or more sensors, devices or systems of a SEE and can include those stored in one or more repository. These features, including sets and subset thereof, may be identified by specific systems, such as a personal physics engine (PPE), where for example movements of a PUM are correlated based on the generated data sets of the SEE. For example, this can include identification of a set of features, for example hand, wrist and arm movements that in aggregate represent the picking up of an object.

In some embodiments, these models can include a Meta-context model, which can include the overall framework for the consideration of the PUM in a SEE and can include specifications from a PUM's HCP (Health Care Profile), specific health, wellness or care conditions or events, for example a hip or knee replacement, medication regimes, including any known effects and the like.

In some embodiments, while a primary focus of these models can be on fall detection, an audio processing pipeline can be extended to monitor other critical events in SEE (e.g., choking, seizures, or cognitive decline). For example, speech coherence analysis (via LLM/LCM) can detect disorientation or confusion, while environmental noise patterns (e.g., silence for >10 minutes) may indicate a medical emergency. These capabilities are integrated into the system's context-aware alerting framework, enabling personalized care plans tailored to each PUM's unique needs.

Each of these models can represent, in part or in whole, the memory of the one or more sensors, devices or systems of a SEE that incorporate one or more AI/ML systems including LLM/LCM. These memories can provide the context for the evaluation of further data sets generated by the SEE and the monitoring of the PUM therein. In some embodiments, such memories may be stored in one or more repository to represent the history of a PUM activities in a SEE. The granularity of the model can be aligned with the granularity and fidelity of the data sets being evaluated by the one or more sensors, devices or systems such that the context for the evaluation and consideration of such data sets is calibrated to the context of that evaluation.

In various embodiments, the environment awareness, pattern, and meta-context models are employed in a hierarchy, which may conserve computing resources. For example, the environment awareness model may identify that the PUM is in a state indicating that the PUM is on the floor, which triggers reconfiguration of the SEE to examine whether the state of the PUM is due to a fall (and may represent an actionable state to notify a stakeholder of) or is due to the PUM deciding to lie down in a controlled manner (e.g., not due to a fall, and therefore may represent an quiescent state to which notify a stakeholder may not be notified of). In this configuration of the SEE, the environmental awareness model may be deactivated (or may remain active to monitor different states of the PUM or SEE) and a pattern model is activated to determine whether the pattern of actions leading to, following from, or both leading to and following from the state of being on the floor represents an actionable pattern of events. For example, the pattern model may identify a change in state from a standing state to a state of lying on the floor occurred with a noise or impact force relative an actionable threshold, within a given time period, or with a series of actions before or after the state indicative of intended motion (e.g., lowering a center of mass towards the floor with support, relaxing while on the floor, looking for an object underneath another object while on the floor, etc.) or unintended motion (e.g., a fall, being in pain after being on the floor, being unconscious while on the floor).

In various embodiments, the SEE may include several pattern models to select between for analyzing various different patterns of behavior, and one or more pattern models are activated based on the type of event detected by the environment awareness model. For example, if a state of the PUM being on the floor is detected by the environment awareness model, pattern models for potential fall analysis, potential seizures, potential cardiac events, may be activated, whereas if a state of the PUM exiting a zone in the SEE is detected by the environment awareness model, pattern models for potential dementia events (e.g., wandering), potential escape events, or potential may be activated

Similarly to an environment awareness model activating one or more pattern models in a hierarchical arrangement for nuanced analysis of the state based on patterns, the one or more pattern models may hierarchically activate one or more meta-context models. In some embodiments, if two or more pattern models indicate for the activation of the same meta-context model, a single instance of the particular meta-context model may be activated or multiple instances (corresponding to the number of pattern models indicating activation) of the particular meta-context model may be activated. In some embodiments, an instance of the meta-context model may not be activated unless a threshold number of pattern models call for the activation of that particular meta-context model. For example, if a pattern model indicates a pattern of a fall event, a meta-context model related to the HCP of the PUM can analyze the results of the pattern model in the context of the known health conditions and previous behaviors of the PUM to determine whether the a PUM is at risk of falls or is engaged in another known behavior that could be interpreted as a fall (e.g., strenuous up-and-down exercises indicated for physical therapy for the PUM), or the like before determining to notify a stakeholder of a fall event. For example, if a pattern model indicates a pattern of wandering (e.g., a potential dementia event occurring), a meta-context model related to the HCP of the PUM can analyze the results of the pattern model in the context of the known health conditions and previous behaviors of the PUM to determine whether the a PUM is at risk or affected by dementia, whether the PUM is known to habitually engage in patterns similar to wandering that are not dementia related (e.g., mild exercise, frequently moving between a kitchen and living room to check a TV program while simultaneously cooking), or the like before determining to notify a stakeholder of a dementia event.

In some embodiments, lower-level models in the hierarchy may be activated by higher level models in the hierarchy to support the determinations and identifications of the higher-level models. For example, a pattern model trained and configured to identify patterns related to a PUM falling may be activated by an environmental awareness model that analyzes sensor data of sounds in the SEE to listen for an impact state indicative of a fall event, but the pattern model may also use image data to analyze the patterns in the determination of fall patterns. According, the system may activate a previously inactive environmental awareness model (and associated environmental sensors to provide the requisite sensor data) trained to analyze images of the environment when the pattern model is activated. In various embodiments, the activation of a same-level or lower-level AI/ML model in the hierarchy may include activating sensors or pulling previously collected, but not transmitted/received data cached in those sensors, as a privacy preserving tool. For example, camera sensors in an environment may be active and collect image data, but locally cache those image data for a time window before deleting or overwriting the image data unless a model reconfigures those cameras to provide the image data in response to detecting an event that allows for the otherwise private data to be transmitted for analysis. For example, when an audio-based model detects a potential fall event, image-based models may request the transmission of the cached image data (and future image data) to evaluate whether a fall event or pattern indicative of a fall occurred, but would otherwise allow the more-sensitive image data to remain private.

Accordingly, by using multiple trained models in a hierarchy, the processing resources of a computing device used to provide the models may be conserved, greater accuracy in the overall output of the models may be provided (e.g., reducing or eliminating hallucinations in the outputs of the individual models to thereby address a computer-centric problem inherent to AI/ML models), privacy and security of the underlying data may be improved, and bandwidth may be conserved in the transmission of notifications to various stakeholders (e.g., not sending notifications for false positives), among other benefits to the systems of the SEE and other devices associated therewith.

A further application of the LLM/LCM or other AI/ML systems can be in the identification or determination of the one or more thresholds that configure the one or more sensors, devices or systems deployed in a SEE. For example, one or more LLM/LCMs can be used to automatically calibrate thresholds, such as decibel levels or accelerometer levels for fall detection, based on historical data and PUM-specific profiles.

In some embodiments, for example where an LCM is employed, the concepts that the LCM recognizes may be formed by multiple sensor data sets from one or more sensors, devices or systems where the LCM, which can be configured to, at least in part, identify the one or more patterns or behaviors represented by the one or more data sets generated by the one or more sensors, devices or systems of the SEE employed to monitor one or more PUM.

As will be appreciated, in an LLM, a “token” is a division of data used for analysis and prediction. In an example using test, the token of “fell” may be predicted as a next token from a series of tokens of individual words of “The PUM stepped on a slippery surface and . . . ”. The present disclosure contemplates that various states or behaviors may be used as tokens in the LLM (rather than or in addition to text representation of the text). In various embodiments, the LLM is configured to check for local coherence for ongoing tokens (e.g., determining whether the next token in a series of tokens is logically appropriate).

As will be appreciated, in an LCM, a “concept” is an atomic idea, which may be represented in various formats. In an example using text, the concept of “falling” may be described in the first sentence of: “the PUM descended from a standing state to a lying down state at a speed and acceleration consistent with gravitational pull” or the second sentence of: “a force of impact on a floor sensor consistent with an object of 50-75 kilograms accelerating at 9.8 meters per second per second over a distance of 1-2 meters has been detected”. The present disclosure contemplates that various states or behaviors may be used as concepts within the LCM (rather than or in addition to text representation of the text). Additionally, an LCM can use various sensor archetypes that are able to represent the same concept (e.g., detection models that are audio based, video/image based, haptic based, etc. and combinations thereof) to provide an initial level of monitoring based on the available or permitted (e.g., according to a privacy profile) and confirm that the same concepts are detected via another monitoring paradigm to reduce or eliminate hallucinations in the models by confirming that the various events are coherent (e.g., congruent) with one another or in series. In various embodiments, the LCM is configured to check for global coherence within a window of ongoing concepts (e.g., determining whether concepts when viewed as a collective series in the window are logically appropriate with one another).

In some embodiments, each of the data streams from the one or more sensors, devices or systems can be evaluated using the one or more feature sets thereof. For example, multi-sensor fusion can combine audio (microphone), motion (accelerometer), and vital signs (heart rate monitor) data into multi-dimensional embeddings, which are segmented for LLM/LCM processing to detect falls with higher accuracy. This fusion can include the use of LLM/LCM or other AI/ML systems, including those specialized for that task. For example, if one or more sensors, devices or systems are configured to identify one or more features, for example, based on thresholds, specific measurements, including changes thereof, identifications of an occurrence, for example a PUM speaking or making noises or other characteristics of the SEE and the PUM therein, these identifications can be identified as features. This feature-identification can include the use of previously identified features, for example those held in a repository or an AI/ML system parameter set, such as, for example, a set of learned NN weights, to aid in the identification or provide a reference from which change and variation may be calculated.

The data sets may comprise multi-dimensional data, for example represented in a topology or other suitable format. In some embodiment, these feature sets may be comprised of data sets from multiple sensors, devices or systems where the aggregate data set or stream is evaluated by one or more systems, including for example LLM/LCM, specialized LLM/LCM or other AI/ML systems, to identify multi-dimensional feature sets that represent, for example a change in state of a PUM in a SEE.

In some embodiments, each of the sensors, devices or systems can be configured to recognize one or more features of the data sets generated by those sensors, devices or systems, and can, for example, represent that feature set and the data thereof in the form of a token or other indicia, including markers, that includes, for example, the identity of the one or more sensors, devices or systems, the data set generated, the one or more features identified or one or more set of metadata, including, for example, time, location, configuration, relationship(s) to other sensors, devices or systems, contextual data, including environmental factors, such as temperature, humidity and the like and other data sets for which such sensors, devices or systems have been configured, where such data sets can be encrypted.

In some embodiments, recognition can include the communication of the feature sets, metadata and other data, for example represented as a token, to one or more LLM/LCM or other AI/ML system with the underlying data sets referenced by such token. This tokenization can aid the efficiency of the LLM/LCM operations, such that the feature sets are evaluated as representations of the underlying data and form the segmentation of such data.

One challenge is the tendency of some LLM/LCM to “hallucinate” and make up answers, such as features, patterns, state changes, threshold breaches or trends and the like. For example, to combat hallucinations, the system can use PPEs and RAGs, which validate LLM/LCM outputs against pre-defined medical rules or historical sensor patterns. In some embodiments, anti-hallucination measures can include the use of PPEs, classifiers where patterns and behaviors have been codified, RAG's and other techniques can be employed to avoid or mitigate such outcomes.

In some embodiments, a frequency of data measurements can be used, at least in part to identify segmentation of these data sets into suitable sets for evaluation and ingestion by one or more LLM/LCM or other AI/ML systems.

In some embodiments, variations in and of the feature sets, including one or more thresholds that, at least in part, determine such features can vary the weighting or vectors of embeddings used by the one or more LLM/LCM, which can include thresholds which can be expressed, for example, in the form of angles, angular momentum or other geometric expressions, including those that involve acceleration, velocity or force in any relationship.

For example, a monitoring system configured to monitor a PUM (e.g., a patient or elderly person), can employ a multi-layered approach, beginning with strategically placed microphones that continuously monitor for acoustic signatures of falls using AI. When a potential incident is detected, the system activates secondary verification through privacy-preserving cameras, millimeter wave radar, or alternative sensors like, for example, thermal imaging or pressure mats to confirm the fall, pinpoint its location, and minimize false alarms.

Once a fall is confirmed, the entire sensor network can be dynamically reconfigured to monitor the PUM's condition while help arrives. For example, after confirmation of a health, wellness or safety event having occurred, the system can reconfigure one or more sensors (e.g., activating pressure mats) and can reroute data streams to focus on vital sign monitoring or injury detection, while LLM/LCMs prioritize alert generation and interface with emergency services or other stakeholders. Some sensors can be activated for the first time at this point (e.g., to begin producing data) or buffered data from some sensors can be gathered and combined with real-time sensor data streams. With this new focus and sensor dataset, sound analysis can shift from trying to identify potential or actual falls to assessing breathing patterns and speech coherence, computer vision analysis can start tracking micro-movements and potential injuries, and mm-wave radar can monitor vital signs-all feeding into an AI orchestration layer, for example, including LLM/LCM, that assesses the person's physical and cognitive state, determines response urgency, and provides continuous updates to emergency services or caregivers while maintaining strict data privacy protocols.

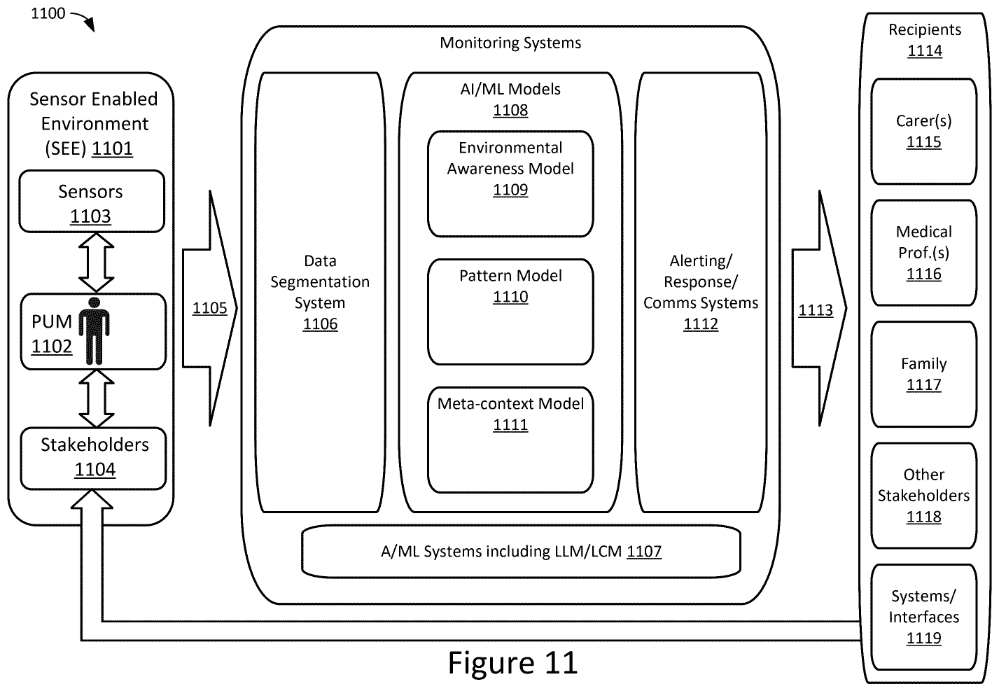

FIG. 11 illustrates a block diagram of an example segmentation flow 1100, in which an example SEE 1101, that can include sensors, devices or systems 1103, one or more stakeholders 1104, and a PUM 1102, where the sensors, devices or systems 1103 generate one or more data sets 1105, according to example embodiments of the present disclosure. These data sets 1105 are ingested by a data segmentation system 1106, as described herein. The data segmentation system 1106 can provide various segments of the data sets 1105 (including all of a data set 1105), amalgamations of two or more data sets 1105 (e.g., at different times, from different SEEs 1101), and supplemental data (e.g., from a database, a HCP, an output of an AI/ML model, or otherwise external to the data set 1105) over time to one or more AI/ML systems 1107, which may include LLM/LCM, and can form part of one or more AI/ML models 1108. In this example, three models are shown, each of which can be configured for differing granularity, fidelity, detail or context. An environment awareness model 1109, a pattern model 1110, and a meta-context model 1111; all have differing contexts supporting the operations of the AI/ML systems 1107 with these segmented data sets, such that the outputs of such AI/ML systems 1107 can be passed to one or more alerting, response or communications systems 1112, which can generate an alert, event, message, specification, instruction or other response or communications 1113 suitable for the intended recipients 1114. The intended recipients 1114 may include, for example, a carer 1115, a medical professional 1116, a family member 1117 or other stakeholders 1118 (including stakeholders 1104 in the SEE 1101 or outside of the SEE 1101) or other systems/interfaces 1119 to, for example, for one or more sensors, devices or systems 103: start/stop, change the configuration, initiate a new process, and the like. Other stakeholders 1118 can include, for example, insurance companies, care facility operators, mobility providers, shopping or other transaction providers, service providers and the like.

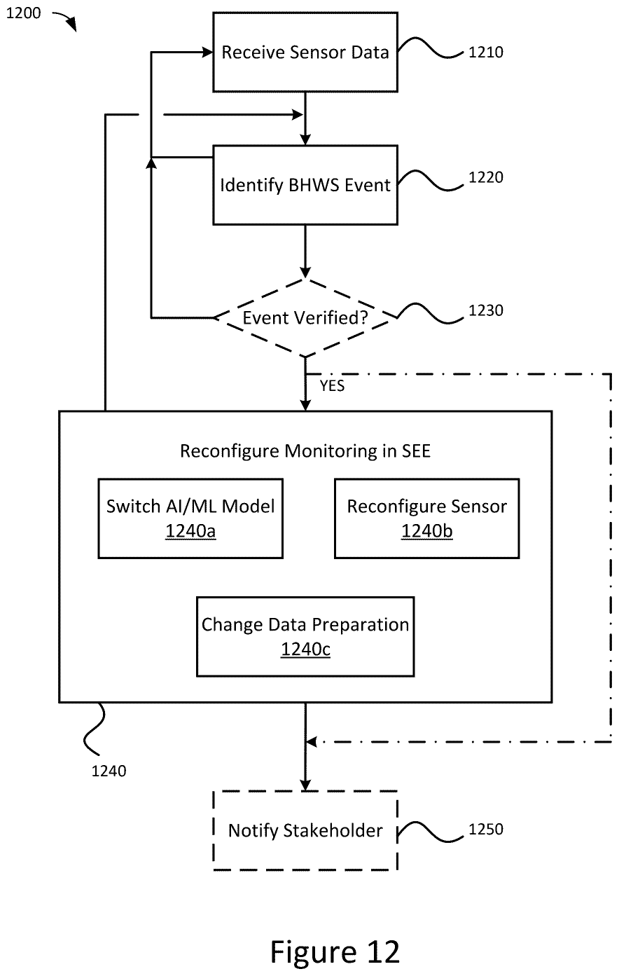

FIG. 12 is a flowchart of an example method 1200 for processing data from a SEE, according to example embodiments of the present disclosure. Method 1200 begins at block 1210, where a computing system that is part of or used in association with a SEE in which a person under monitoring (PUM) is monitored receives sensor data from the SEE. The SEE includes a plurality of environmental sensors, which may include sensors worn, carried, or implanted in the PUM and sensors, and sensors disposed in the SEE that are not worn, carried, or implanted in the PUM, all of which may provide various data at various rates, frequencies, granularities, encodings, formats, and encryptions, which one or more artificial intelligence or machine learning (AI/ML) models use to monitor the health, wellness or safety of the PUM and other parties in the SEE.

At block 1220, the AI/ML model analyzes the sensor data to identify a behavioral, health, wellness, or safety (BHWS) event occurring in the SEE affecting the PUM. If no BHWS events are identified, method 1200 may repeat analysis of the sensor data, or a new window of sensor data, per block 1220 until a BHWS event is identified. In various embodiments, reconfiguring the SEE from a first configuration to a second configuration is based on the first BHWS event so that different data sharing policies associated with different types of BHWS event, can be used to select how the change the configuration of the AI/ML models and sensors, such as a data sharing policy associated with the type of the BHWS event to preserve privacy, reduce overall processing needs, reduce overall bandwidth needs, or the like and combinations thereof.

In some embodiments, identifying the BHWS events includes detecting a state of the PUM or the SEE, and analyzing the state using an Environment Awareness Model. In some embodiments, identifying the BHWS event includes detecting a series of states of the PUM or the SEE via the Environment Awareness Model, and analyzing the series of states using a Pattern Model. In some embodiments, identifying the BHWS event includes detecting a behavioral pattern via the analyzed series of states, and analyzing the behavior using a meta-context model in comparison to at least one of a health care profile (HCP), model in a personalized physics engine (PPE), or a learned habitual behavior of the PUM.

Examples of different BHWS events that may lead to a reconfiguration of the SEE may include quiescent events and actionable events. In various embodiments, the quiescent events represents the behaviors and movement frameworks that do not cause the system to generate an alert or other follow-up action (e.g., an actionable event) to a stakeholder, but may still cause the system to adjust how data are collected and analyzed. For example, a behavior of a PUM sitting down to watch TV at three in the afternoon may be considered a quiescent event for the PUM whereas sitting down to watch TV at three in the morning may not be considered a quiescent event for the PUM based on the observed wakefulness patterns for the PUM (e.g., indicative of a disrupted sleep pattern). For example, a behavior of a PUM vigorously using a stationary bicycle at nine in the morning may be considered a quiescent event for the PUM whereas the PUM lackadaisically using a stationary bicycle at nine in the morning may not be considered a quiescent state for the PUM based on the observed exercise patterns for the PUM (e.g., indicative of poor health preventing vigorous exercise). For example, a behavior of a PUM more noisily or forceful impacting a surface than a threshold deviation from previous noises or forces of impact with that surface may be indicative of a fall or arrested fall, which the system may treat as a non-quiescent behavior for which follow-up actions are initiated. In each of the examples, the quiescent or actionable event may constitute a BHWS event as the system may activate different sensors or AI/ML models as the system has identified an event that may require further analysis to continue monitoring the PUM or SEE (e.g., when the PUM quiescently moves from one room to another) or to gather more information to determine whether the event is actionable or quiescent, or whether the event actually occurred or was a hallucination of the AI/ML model.

At block 1230, the system (optionally) verifies the identification of the BHWS event identified per block 1220. In various embodiments, as part of performing block 1230, the system reconfigures the sensors or AI/ML model used per block 1240 to retrieve more or different data or analyses of the data. In various embodiments, successful verification of the BHWS event is used as a prerequisite for notifying a stakeholder to the BHWS event so as to reduce the number of false positives that the stakeholders are made aware of and to reduce the associated bandwidth for transmitting those notifications.

In some embodiments, the system simulates the SEE and the PUM and uses a Large Language Model or Large Context Model artificial intelligence or machine learning (LLM/LCM AI/ML) system to predict a future BHWS event via tokens or concepts of previous BHWS events included in the sensor data, and compares the predicted event to identified events. When the predicted and identified events match, the identification of the BHWS event is then verified, but when the predicted and identified events do not match, the system may take further action to verify or confirm what the actual event is compared to the identified event (e.g., adjusting sensors, models, etc.).

In some embodiments, the system uses a Large Language Model or Large Context Model artificial intelligence or machine learning (LLM/LCM AI/ML) system to identify that a first identified BHWS event is incongruous with a second identified BHWS event according to tokens or concepts represented by the identified BHWS events. For example, if a first identified BHWS system represent the concept of the PUM exercising, and the second identified BHWS event is of the PUM sleeping, a timing of the two events may make the overall concept incongruous as the actual events may be (per global coherency) that the PUM was first rolling while sleeping and then returned to restful sleeping or that the PUM was exercising and is resting or was injured while exercising. When the events display coherence with one another, the identification of the BHWS event is then verified, but when the events are incongruent and lack coherency with one another, the system may take further action to verify or confirm what the actual event is compared to the identified event (e.g., adjusting sensors, models, etc.).

In some embodiments, the system uses a personalized physics engine (PPE) to model the physical capabilities of the PUM, and compares the behaviors indicated in the identified BHWS events against the model to determine whether the PUM is physically capable for performing the identified behaviors and verify the identification. When the model of the PUM is not capable (without injury) of performing the identified events, the system may determine that the identified events were hallucinations, and the system may take further action to verify or confirm what the actual event is compared to the identified event (e.g., adjusting sensors, models, etc.)

At block 1240, the system reconfigures how the SEE monitors the PUM from a current (e.g., first) configuration to an updated (e.g., second) configuration based, at least in part, on the BHWS event detected per block 1220. Method 1200 may return to block 1220 to continue analyzing the sensor data according to the updated (e.g., second) configuration to provide further analysis of the PUM or SEE or confirm an earlier analysis (e.g., a first BHWS event) by using only the sensor data received after the reconfiguration, a combination of the sensor data received before and after the reconfiguration, a combination of the sensor data received only before the reconfiguration and external data (e.g., quiescent state data, data from an HCP, etc.), a combination of the sensor data received only after the reconfiguration and external data, a combination of the sensor data received before and after the reconfiguration and external data, or the like.

In various embodiments, reconfiguring the SEE from a first configuration to a second configuration can include one or more reconfiguration actions, which may include: (1240a) switching the AI/ML model to a second AI/ML model to analyzing the sensor data with the second AI/ML model; (1240b) reconfiguring at least one of the plurality of environmental sensors to receive future sensor data according to a second configuration from at least one of environmental sensors that has been reconfigured; and (1240c) changing how data received from individual environmental sensors in the plurality of environmental sensors are prepared for analysis by the AI/ML model.

In various embodiments, reconfiguring the SEE by switching the AI/ML model used to analyze the first sensor data to the second AI/ML model to analyze the second sensor data includes using a hierarchical activation scheme in which a first model used is selected from a group of Environment Awareness Models, Pattern Models, and meta-context model as the AI/ML models to process earlier sensor data and select a different second model from that group to analyze later sensor data (or various combinations of later sensor data, earlier sensor data, and external data). In various embodiments, the selection of the second AI/ML model is based on a type of the BHWS event detected. In some embodiments, more than one subsequent model may be activated based on the outputs of the first model or the inputs used by the second model (e.g., to provide data or analysis for the second model) or based on the outputs of the second model (e.g., to activate a next model in the hierarchy).

In various embodiments, reconfiguring an environmental sensors can include sending a configuration command to one or more of a plurality of environmental sensors to change a configuration of one or more environmental sensors by: activating at least one of the plurality of environmental sensors that is currently inactive; deactivating at least one of the plurality of environmental sensors that is currently active; increasing a granularity of data collected by at least one of the plurality of environmental sensors that is currently active (e.g., to report more data in a particular time period, report cached data); decreasing the granularity of data collected by at least one of the plurality of environmental sensors that is currently active (e.g., to report less data in a particular time period); increasing a reporting rate of at least one of the plurality of environmental sensors that is currently active; decreasing the reporting rate of at least one of the plurality of environmental sensors; changing an optical focus of at least one of the plurality of environmental sensors that is currently active (e.g., activate a motor to move where a camera is pointed in the SEE, activate a hardware zoom-in/zoom-out function).

In various embodiments, reconfiguring how data are received, amalgamated, or segmented from individual environmental sensors of the plurality of environmental sensors for analysis by the AI/ML model can include altering a segmentation scheme for the data. In some examples, the alteration of the segmentation scheme can include: identifying different features from the updated/second sensor data that are not identified from (but may be present in) the earlier/first sensor data, such as looking for different event cues within the full data set; analyzing longer segments of the second sensor data compared to the first sensor data (e.g., for concepts vs. tokens, data windows of X duration); analyzing shorter segments of the second sensor data compared to the first sensor data (e.g., for tokens vs. concepts, data windows of X duration); incorporating additional data from a second environmental sensor of the plurality of environmental sensors with the second sensor data that was not incorporated with the first sensor data (e.g., multi-dimension fusion of data and analyses); and combinations thereof.

At block 1250, the system (optionally) notifies a stakeholder for care of the PUM of the one or more BHWS events identified in the SEE that affect the PUM. In various embodiments, the notification is tokenized, which can include a first level of information that is accessible via a first decryption scheme (including plaintext) and second or subsequent levels of information that are accessible via various different decryption schemes to allow the system to share information with multiple parties quickly and control the dissemination of that information on a need-to-know basis to the parties.

For example, when notifying a stakeholder, the system may generate a first token that includes a type of the first BHWS event in an unencrypted format and a segment of the sensor data used by the AI/ML model to identify the first BHWS event or other information pertinent to the BHWS event in an encrypted format when transmitting the first token to a first external system in which the first external system has (or is provided by the system) with a corresponding first decryption scheme (e.g., a key) to access the segment of the first sensor data. That same first token may be shared by the system to a second stakeholder, or by the external system to a second stakeholder, but once transmitted is no longer under the control of the system. Accordingly, additionally or alternatively to generating a second token with data included in a payload for the second stakeholder, the system can include a second segment of sensor data or other pertinent information in the first token, and control access by applying a second encryption schemes to the second segment. Accordingly, the token may be provided to devices or systems associated with various stakeholders for the care of the PUM as well as to record keeping systems (e.g., a host of a distributed ledger, blockchain ledger or other immutable record) with control over continued access to the sensitive data included therein.

Accordingly, one token can be transmitted to a first external system and to a second external system such that the second external system is provided a decryption schema for one of the encrypted formats of data that is not provided to the first external system (and vice versa). Additionally or alternative, two tokens can be transmitted to a corresponding one of first external system and to a second external system such that the data are encrypted and selected for use by the respective external systems and associated stakeholders.

The particular stakeholders selected to receive the notification may be based on the HCP or a privacy profile for the PUM, a type of event that has occurred, and a last time that the system attempted to notify a stakeholder (e.g., escalating notification when earlier stakeholders have not responded or arrived). In various embodiments, the stakeholder device or system is associated with a stakeholder for care of the PUM selected from a group of potential stakeholder that may include one or more of: the PUM; a caregiver of the PUM; a friend of the PUM; a neighbor of the PUM; a family member of the PUM; an insurance provider for the PUM; a medical professional; and an emergency responder.

In some embodiments in which the system models predicted events, the system may preemptively configure the SEE to capture the actual occurrence of that event. In such embodiments, the system compares a predicted event against a model of physical capabilities of the PUM in a personalized physics engine (PPE) and, in response to determining that at least one behavior included in the predicted event is within the physical capabilities of the PUM according to the model in the PPE, selects the second configuration of the SEE to capture data in a format for recording an actual occurrence of the predicted event. The system may also preemptively notify a stakeholder before the actual event is predicted to occur based on the predicted event.

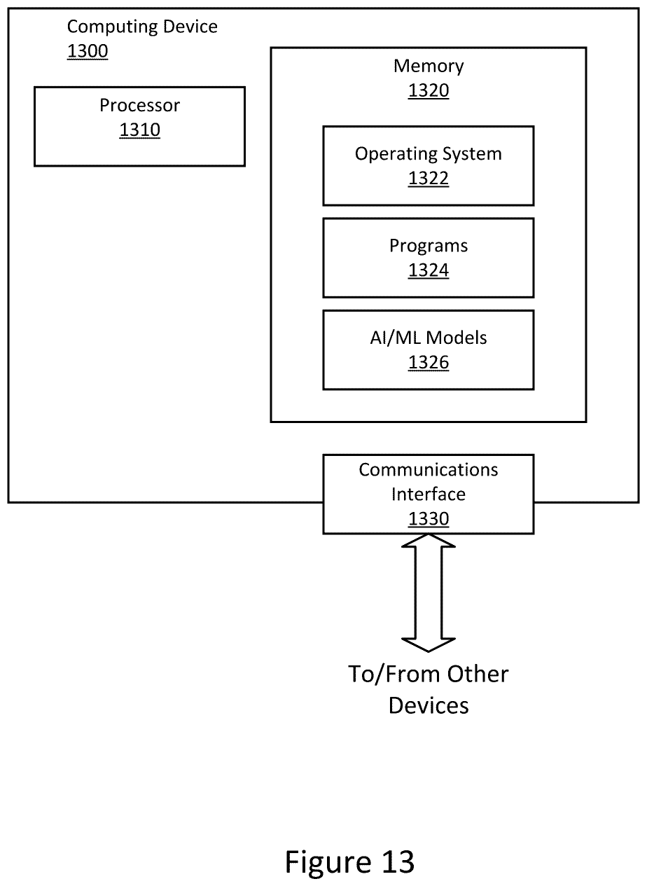

FIG. 13 illustrates an example computing device 1300, as may be used as a controller in a SEE to monitor a PUM, as part of a sensor monitoring a PUM, as part of a central or distributed service providing calibration systems for generating and curating AI/ML models for distribution to the SEEs, and the like, according to embodiments of the present disclosure. For example, the computing device 1300 may perform the operations set out in one or more of methods 800, 900, or 1000. The computing device 1300 may include at least one processor 1310, a memory 1320, and a communication interface 1330.

The processor 1310 may be any processing unit capable of performing the operations and procedures described in the present disclosure (e.g., methods 800, 900, 1000, 1200). In various embodiments, the processor 1310 can represent a single processor, multiple processors, a processor with multiple cores, and combinations thereof.

The memory 1320 is an apparatus that may be either volatile or non-volatile memory and may include RAM, flash, cache, disk drives, and other computer readable memory storage devices. Although shown as a single entity, the memory 1320 may be divided into different memory storage elements such as RAM and one or more hard disk drives. As used herein, the memory 1320 is an example of a device that includes computer-readable storage media, and is not to be interpreted as transmission media or signals per se.

As shown, the memory 1320 includes various instructions that are executable by the processor 1310 to provide an operating system 1322 to manage various features of the computing device 1300 and one or more programs 1324 to provide various functionalities to users of the computing device 1300, which include one or more of the features and functionalities described in the present disclosure (e.g., method 800, 900, 1000, 1200). One of ordinary skill in the relevant art will recognize that different approaches can be taken in selecting or designing a program 1324 to perform the operations described herein, including choice of programming language, the operating system 1322 used by the computing device 1300, and the architecture of the processor 1310 and memory 1320. Accordingly, the person of ordinary skill in the relevant art will be able to select or design an appropriate program 1324 based on the details provided in the present disclosure.

Additionally, the memory 1320 may include one or more AI/ML models 1326 that interact with, are trained by, or are curated by the programs 1324. The AI/ML models 1326 may include linguistic AI/ML models that are available for use to various SEEs as a hub AI from a central service as well as localized instances thereof for use as “edge” AI/ML models that are adjusted to reflect localized conditions in a particular SEE to track and monitor a PUM, as described herein.

The communication interface 1330 facilitates communications between the computing device 1300 and other devices, including sensors in a SEE, which may also be computing devices as described in relation to FIG. 13. In various embodiments, the communication interface 1330 includes antennas for wireless communications and various wired communication ports. The computing device 1300 may also include or be in communication, via the communication interface 1330, one or more input devices (e.g., a keyboard, mouse, pen, touch input device, etc.) and one or more output devices (e.g., a display, speakers, a printer, etc.).

Although not explicitly shown in FIG. 13, it should be recognized that the computing device 1300 may be connected to one or more public or private networks via appropriate network connections via the communication interface 1330. It will also be recognized that software instructions may also be loaded into a non-transitory computer readable medium, such as the memory 1320, from an appropriate storage medium or via wired or wireless means.

Systems, methods, and apparatuses of the present disclosure may be implemented on a variety of devices, such as but not limited to IPUs, DPUs, CPUs, GPUs, ASICs, FPGAs, DSPs, or any other device capable of processing data. Instructions for performing the same may be provided as hardware or firmware on any computer-readable medium including volatile and non-volatile forms of memory. Particular implementations of techniques of the present disclosure may be structured in any number of ways, including but not limited to a modular program architecture, a monolithic program architecture, on a single device, and distributed across more than one device or processor.

Systems, methods, and apparatuses of the present disclosure may be implemented on a variety of devices, such as but not limited to IPUs, DPUs, CPUs, GPUs, ASICs, FPGAs, DSPs, or any other device capable of processing data. Instructions for performing the same may be provided as hardware or firmware on any computer-readable medium including volatile and non-volatile forms of memory. Particular implementations of techniques of the present disclosure may be structured in any number of ways, including but not limited to a modular program architecture, a monolithic program architecture, on a single device, and distributed across more than one device or processor.

Although certain figures and descriptions have been provided, many additional variations and modifications will be apparent to those of skill in the art. It will be appreciated that presenting all possible variations and modifications is an impractical task, and thus any sequence, particular structural or device implementation, or underlying technique of the present disclosure may be substituted or modified to meet the needs of particular implementations, and that doing so may not depart from the scope of the present disclosure. It will therefore be appreciated that the examples presented herein are presented for illustrative purposes only, and are in no way intended to be limiting of a scope of the present disclosure. It will also be apparent to any individual of skill in the art that various embodiments described herein and elements thereof may be combined as needed to suit any particular implementation, and that doing so does not depart from the scope of the present disclosure. As such, the scope of the present disclosure is not to be understood as being limited by the figures or specification presented herein; the scope of the present disclosure should instead be understood in a context of the appended claims and their equivalents.

Certain terms are used throughout the description and claims to refer to particular features or components. As one skilled in the art will appreciate, different persons may refer to the same feature or component by different names. This document does not intend to distinguish between components or features that differ in name but not function.

As used herein, the term “optimize” and variations thereof, is used in a sense understood by data scientists to refer to actions taken for continual improvement of a system relative to a goal. An optimized value will be understood to represent “near-best” value for a particular reward framework, which may oscillate around a local maximum or a global maximum for a “best” value or set of values, which may change as the goal changes or as input conditions change. Accordingly, an optimal solution for a first goal at a particular time may be suboptimal for a second goal at that time or suboptimal for the first goal at a later time. Similarly, terms of “minimize” and “maximize” shall generally be understood to refer to optimizing for a “best” lowest value or highest value, respectively, and may include the identification of local minima, local maxima, a global minimum, or a global maximum, which can vary at different times or under new goals or conditions.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, the term “or” is to be interpreted in the inclusive sense and not the exclusive sense unless explicitly stated otherwise or when clear from the context. Accordingly, recitation of “A or B” is intended to cover the sets of A, B, and A-B, where the sets may include one or multiple instances of a particular member (e.g., A-A, A-A-A, A-A-B, etc.) and any ordering thereof.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a particular member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, identifying, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

What is claimed is:

1. A method, comprising:
receiving first sensor data from a sensor enabled environment (SEE) in which a person under monitoring (PUM) is monitored, wherein the first sensor data are received from a first configuration of the SEE, wherein the SEE comprises a plurality of environmental sensors and an artificial intelligence or machine learning (AI/ML) model;
identifying, via the AI/ML model analyzing the first sensor data, a first behavioral, health, wellness, or safety (BHWS) event occurring in the SEE affecting the PUM;
in response to identifying the first BHWS event, reconfiguring how the SEE monitors the PUM from the first configuration to a second configuration based on the first BHWS event;
receiving second sensor data from the SEE according to the second configuration; and
identifying, via analysis of the second sensor data, a second BHWS event occurring in the SEE affecting the PUM.

2. The method of claim 1, wherein reconfiguring the SEE from the first configuration to the second configuration includes performing a reconfiguration selected from the group consisting of:
(A) switching the AI/ML model to a second AI/ML model; and
analyzing the second sensor data with the second AI/ML model as part of identifying the second BHWS event;
(B) reconfiguring at least one of the plurality of environmental sensors, wherein the second sensor data are received, from the SEE, at least in part, using the at least one of environmental sensors that has been reconfigured; and
(C) changing how data received from individual environmental sensors in the plurality of environmental sensors are prepared for analysis by the AI/ML model.

3. The method of claim 2, wherein switching the AI/ML model used to analyze the first sensor data to the second AI/ML model to analyze the second sensor data comprises:
using a first model from a group consisting of an Environment Awareness Model, a Pattern Model, and a meta-context model as the AI/ML model to process the first sensor data; and
using a second model, different from the first model from the group consisting of the Environment Awareness Model, the Pattern Model, and the meta-context model to process the second sensor data;
wherein the second one of the Environment Awareness Model, the Pattern Model, and the meta-context model is selected based on:
a type of the BHWS event detected.

4. The method of claim 2, wherein reconfiguring the at least one of the plurality of environmental sensors includes sending a configuration command for the at least one of the plurality of environmental sensors from the group consisting of:
activating the at least one of the plurality of environmental sensors;
deactivating the at least one of the plurality of environmental sensors; and
increasing a granularity of data collected by the at least one of the plurality of environmental sensors;
decreasing the granularity of data collected by the at least one of the plurality of environmental sensors;
increasing a reporting rate of the at least one of the plurality of environmental sensors;
decreasing the reporting rate of the at least one of the plurality of environmental sensors; and
changing an optical focus of the at least one of the plurality of environmental sensors.

5. The method of claim 2, wherein reconfiguring how data received from individual environmental sensors of the plurality of environmental sensors are amalgamated for analysis by the AI/ML model in the second sensor data relative to the first sensor data is selected according to a segmentation scheme selected from the group consisting of:
identifying second features from the second sensor data that are not identified from the first sensor data, wherein the second features are present in the first sensor data;
analyzing longer segments of the second sensor data compared to the first sensor data;
analyzing shorter segments of the second sensor data compared to the first sensor data; and
incorporating additional data from a second environmental sensor of the plurality of environmental sensors with the second sensor data that was not incorporated with the first sensor data.

6. The method of claim 1, wherein reconfiguring the SEE from the first configuration to the second configuration based on the first BHWS event includes:
wherein different data sharing policies are associated with different types of BHWS event, the method further comprising:
identifying a type of the BHWS event detected; and
selecting the second configuration according to a data sharing policy associated with the type of the BHWS event.

7. The method of claim 1, wherein identifying the first BHWS event further comprises:
detecting a state of the PUM or the SEE; and
analyzing the state using an Environment Awareness Model.

8. The method of claim 7, wherein identifying the first BHWS event further comprises:
detecting a series of states of the PUM or the SEE via the Environment Awareness Model; and
analyzing the series of states using a Pattern Model.

9. The method of claim 1, wherein identifying the first BHWS event further comprises:
detecting a behavioral pattern via the analyzed series of states; and
analyzing the behavior using a meta-context model in comparison to at least one of a health care profile (HCP), model in a personalized physics engine (PPE), or a learned habitual behavior of the PUM.

10. The method of claim 1, further comprising:
using a Large Language Model or Large Context Model artificial intelligence or machine learning (LLM/LCM AI/ML) system to predict a future BHWS event via tokens or concepts of previous BHWS events included in the first sensor data and the second sensor data.

11. The method of claim 1, further comprising:
using a Large Language Model or Large Context Model artificial intelligence or machine learning (LLM/LCM AI/ML) system to identify that the first BHWS event is incongruous to the second BHWS event according to tokens or concepts represented by the first BHWS event and the second BHWS event with respect to a quiescent state of the PUM or SEE.

12. The method of claim 1, further comprising:
comparing the first BHWS event against the second BHWS event to confirm whether the first BHWS occurred or is a hallucination; and
in response to confirming via identification of the second BHWS event that the first BHWS actually occurred, transmitting a notification to a stakeholder for care of the PUM that identifies occurrence of the actual event.

13. The method of claim 1, wherein the first BHWS event is a predicted event generated by the AI/ML model, further comprising:
comparing the predicted event against a model of physical capabilities of the PUM in a personalized physics engine (PPE);
in response to determining that at least one behavior included in the predicted event is within the physical capabilities of the PUM according to the model in the PPE:
transmitting a notification to a stakeholder for care of the PUM that identifies the predicted event; and
selecting the second configuration of the SEE to capture data in a format for recording an actual occurrence of the predicted event.

14. The method of claim 1, wherein the first BHWS event is a predicted event generated by the AI/ML model, further comprising:
comparing the predicted event against a model of physical capabilities of the PUM in a personalized physics engine (PPE); and
in response to determining that at least one behavior included in the predicted event is outside of the physical capabilities of the PUM according to the model in the PPE, classifying the predicted event as a hallucination of the AI/ML model.

15. The method of claim 1, further comprising:
generating a first token that includes a type of the first BHWS event in an unencrypted format and a segment of the first sensor data used by the AI/ML model to identify the first BHWS event in an encrypted format; and
transmitting the first token to a first external system.

16. The method of claim 15, wherein the external system is selected from the group consisting of:
a distributed or blockchain ledger; and
a stakeholder device or system.

17. The method of claim 16, wherein the stakeholder device or system is associated with a stakeholder for care of the PUM selected from the group consisting of:
the PUM;
a caregiver of the PUM;
a friend of the PUM;
a neighbor of the PUM;
a family member of the PUM;
an insurance provider for the PUM;
a medical professional; and
an emergency responder.

18. The method of claim 15, further comprising:
transmitting the first token to a second external system, wherein the second external system is provided a decryption schema for the encrypted format that is not provided to the first external system.

19. The method of claim 15, further comprising:
identifying a second external system associated with a second decryption schema based on the type of the first BHWS event and a type of the second BHWS event;
generating a second token that includes the first type of the first BHWS event and a second type of the second BHWS event in the unencrypted format and a second segment of the second sensor data used by the AI/ML model to identify the second BHWS event in a second encrypted format decryptable according to the second decryption schema; and
transmitting the second token to a second external system.

20. A system, comprising:
a processor;
a memory, including instructions that, when executed by the processor perform operations including:
receiving first sensor data from a sensor enabled environment (SEE) in which a person under monitoring (PUM) is monitored, wherein the first sensor data are received from a first configuration of the SEE, wherein the SEE comprises a plurality of environmental sensors and an artificial intelligence or machine learning (AI/ML) model;
identifying, via the AI/ML model analyzing the first sensor data, a first behavioral, health, wellness, or safety (BHWS) event occurring in the SEE affecting the PUM;
in response to identifying the first BHWS event, reconfiguring how the SEE monitors the PUM from the first configuration to a second configuration based on the first BHWS event;
receiving second sensor data from the SEE according to the second configuration; and
identifying, via analysis of the second sensor data, a second BHWS event occurring in the SEE affecting the PUM.
